SEQUENCE LISTING (2 sequences)

SEQ ID NO: 1 - DNA, 2062 bp, Homo sapiens
agcggaagggacagattggatcgcacctgtcacagccacgctctggaatagcgagggcccagcgtccccaggcccagggtgaggagaatccctgccacactgtgtctggaattggtgggttcttggtctcactgacttcaagaatgaagccgcggaccctcacgaaccctctgtggaagcggtggtctgacacaagcatttctggatgacgctgaacctgcttacaactccctgctgctgtcaggcagaacattctccaaaccgcatgaatttggaagtctggacacttcaggcatcatcaggtttatggttttgtcataaccctgctgaaagtcctttaccatctccaggcctccatttattttctataaaataaggagtttggaatggcactgtgttgatattctccagttttgtgaaacatcgtactcaacaatcgcgagcaggcattttacagaccttgctttcctagacctgctcaagtagaacattcaggtcagtgctaagatttaatatgctgcctccgtctgacctccacattatggaaaaaaatataaaggaaagagcaaaaggtagagcaagtaggtttcgggcttggtggagtagctctgagtgctgccctgtccttggctgtgtgtgccaggtgaagcgtctgtgaaggtccccagtggggtgtgcagagcacaagggctgcttctgaatgcaggcacgtgatctaaggacactgtgcatcaccagtgtgaccccaggagtagtttcagagctttgataagacctaggccgggctcagtggctcacgcctgtcatctcagcactttgggaggccgaggtgggtggatcagctgaggtcgggagttcgagaccagcctgaccaatatggtgaaatcctgtccctactgaaagtacaaagattggttgggtgtgggggcgagcgcctgtggtcccagctacaccacaggaatctgggctgtaaaggcgggatataaattcaagtccacatgctatggagaaatcagaggtttcgaaagtatctccgtgactgtttttgaagtcacagctaataattcaccgtctgacactccggggcttgcctggatctaaaaatagttgttcccccgagctggtcggggaactggctgagaagggagggagctgcgtggaacacattctgaaccatcaggaaacccaaagcaaaatcgaatcttgccacctaagatttgattgttaaaagatggatttgacagctgattccccattaattacatttgtaaagatatttgaaagacaggaatgaaatgacagtggccctgacacagtccttggggtttaagacggagtctgacattgtatctgtctacggttctgcagcagcttagcgacatcagagacaattgaagcaaagggcccacttatggctgacagctgaccctgcagcccagctgggaagcccctgagacgacccgggtccagccatcagggaagggaaccagcgtggccccaggagggctttaggctgaggcttctcccaggatgtggaggctcagcactttgcacaaattggattcatgccaaaggaaactgaaagcctgcctttctttttttcccagtgcacatctcagattatttggcctttgtccgaggactgaaaacagttctgtgtccaagtatgtttttaatacctgatatttatttcacaaaaaaactgaaattgctttgtgtgtccaggcttgaatgtttaaggcatacttgattaatacatgtgtgctgagtgcttcctggggctcctagacactgccaaaggtagctcagtggggaaattcaaggcaattttccccttaacttttgctacttctgaccattttccacttatgttattatgcttctgtatccatctgtctgcctatgtctagctatccatcatctatcaatcaatcactctatcttgcaatggaggaattaactgtgatgttttcaagcgtgtcgcttctctcatattcctattaacatattggaaacgctcagctaaatagaataaagtttttagcccaaaaaaaaaaaaaaa

SEQ ID NO: 2 - DNA, 10002 bp, Homo sapiens
agaatcgaaactgagagctcctgggcaggctcggcagggcaggcagctccaggagggcttcgaaccgtggccaacagttccagtggactgcgtggacccgtgagctcaggagcctcagacgcctccctggagagccaagctggtgttcgaggtggcgcctccagggtccaccctgctgcccaacagccccgcggccaccagagggccgtccctggcccggctgtgtgccctggtggacctgtgtctgggctgctcccgctgcacccagcggctcaatgaaagcacctacgtcctccgtagggtggagcatgactgctcccgcgagatcctgctggcccgctttaagcaggccaccaagagcaaggtctggcgcgtggtgggctgccggcccaccttcccaaggcccctgtgctaccaagtctgccactactacagccctgggctcggctgccggcgccaccgaaaccggtgcacctttgcccgcagtcgcgaggaggccctggtctggaccttcgagcgtcagcacaacctccagcgcctatggctgaaggcggaggtgcagggcagcggggcccagggaggggcaggccgggcggccgacgccatccttacggagtttggcggccgcttcgagctgctttgctccctctgcttcaggcgctgtcccccatgcatctgtcgcgtggacccccaggggcagtgccctgagcacggagcctgcccctccctcctggcccacgtgagcgccgagggccgccgcaagcaacagtttgtggtggtgaggccgcggccccgggccggccagcctcctgcctactgcaggtttgtggggcgtgggcagccgtgctggcgtggggagtcccgctgccagtttgcacacagcgccgtggagatggctgtgtgggaggccgagcagctgggtggcctccagcggggggacctgctcacaccccctgcccctgatggcgacgggcgcacggccccccttggccagccccctggggcccagctgtactgcccggcctgcttggtcacctgccactctcaggaggccttcgagaaccactgcgcatcctcggagcacgcacagatggtggccttcgaccaggccctgccctgggagcaccgttccccacccccgggactctccaagttcgagctctgcccaaagcctgacctctgtgagtatggggacgcctgcaccaaggcacactcagcacaggagctgcaggagtgggtccggcgcacgcaggctgtggagctgcgggggcaggcggcctggcaggacgggctggtgccctaccaggagcggctgctggccgagtaccagcgcagcagcagtgaggtccttgtgctggcagagacccttgatggagtgcgtgtcacctgcaaccagcccctgatgtaccaggcccaggagaggaagacccagtacagctggacgtttgccgtccactctgaggagcccctgctacacgtggccctgctgaagcaggagccaggagccgacttctctctggtggctcccggcctcccgccaggccggctctacgcacggggtgagcgcttccgtgtgcccagctccactgccgacttccaggtgggagtgcgtgtgcaggctgcctcgttcggcacctttgagcaatgggtggtcttcgactttggccgccggccggtgctgctacaaaagctggggctgcagctgggccaggggcgtcgcccaggaccctgcaggaatctggcgctcggccaccctgaggagatggagcgctggcacactggcaaccgccacgtggtgcctggcgtggagcggacggccgagcagacggccctgatggccaagtacaagggccctgccctggccctggagttcaaccgcagcagcgtggcctcgggccccatctcaccaaccaactatcggcagaggatgcaccagtttctctatgaggaggaggcggctcagcagcagctggtggccaagctgaccctgcggggccaggtgttcctgaagacggcattgcagacgccagcgctgaacatgctcttcgcgcctccgggagcactgtacgcagaggtccccgtcccctcctccctgatgccagacacagaccagggcttcctgctgggccgggcggtcagcacagccctggtggcccctgtacctgcacccgacaatacggtgttcgaggtgcggctggagaggcgggccagctcagagcaggcgctgtggctgctgcttccggcccgctgctgcttggccctggggctgcagcctgaggcccgcctggtcctggaggtgcagttccagattgacccgatgaccttccgcctctggcaccaggcagtggacacactgcctgaggagcagctggtggtgcccgacttgcccacctgcgccctgcccagaccttggtctgtcccacccttgcggcgtggcaaccgcaagcaggagctggccgtggcgctcatcgcgggctggggccctggggatgggaggcgtgtccccccgctactcatctatggcccctttggcaccggcaagacctacacgctggccatggcctccctggaggtcatccggaggcctgaaaccaaggtgctcatctgcacacacaccaacagtgccgccgacatctacatccgggagtatttccacagccacgtcagcggcggccaccccgaggccactcctctccgtgtgatgtacacggaccggccgctgagccagacggacccagtcacgctgcagtactgttgcctgaccgacgaccgccaggctttccgcccgcccacacgggcagagctggcgcggcaccgcgtggtggtcaccaccacctcccaggcccgtgagctcagggtgccggtcggcttcttctcccacattctcatcgatgaggcggcccagatgctggagtgcgaggccctcaccccgctggcctatgcctcgcacggcacccgcctcgtgctggcgggcgaccacatgcaggtcacaccccggctgttcagtgtggccagggcccgggcggccgagcacacgctgctgcaccgcctcttcctgtgctaccagcaggagactcacgaggtggcgcggcagagccgcctggtcttccacgagaactaccgctgcacggacgccattgtcagcttcatctcgcggcacttctacgtggccaagggcaaccccatccacgccaggggcaaggttccgccccacccccggcactacccgctcatgttctgccacgtggcgggcagcccagaccgggacatgtccatggcgtcctggctgaatctggctgagattgcgcaggtcgtcgagaaggtgcaggaggcctacaacacctggcccagctgctggggcggccgcgagcagaggtgcatctgtgtcgtttcccacggtgcccaggtcagtgcactgaggcaggagctgaggaggcgggacctaggccaggtgtctgtcggcagttttgagatcctgccagggcggcagttccgggtcgtggtgctcagcactgtgcacacctgccagagcctgctcagccctggggcactggcccctgagttcttcaccgacgcccgcgtgctcaacaccgtcctgacccgcgcccagtcccagctggtggtagtgggggacgccgtggccctctgctccttcggggcctgcggcaagctctgggagagcttcatccgtgagtgcgtggagcggcacagtgtctgccccgagggcctgtccatggagcaggtcgagcagggtgtggcgcagagacggcgctggcctccccgaggcacacaggctggggcagcggggaactgggaggctgccccagagccagtaggggacctggccgaggagcaggcggctgtggtgacggccatggtgaaggcagagccgggagatgaggctctgagcccagcatcccgtgacatcacggcaaccacagcgcagacggaggctgcggcagcaccagcaggagacgcagtgaaggaagacgtggtgcccggggcctgtgcggcaggagcggctgctgcagcgggcgtggagtccacggaggctgaggatgcagaggctgacttctggccgtgggacggggagctcaacgctgacgacgccatcctacgggagcttctggacgagagccagaaggtgatggtgaccgtcggggaggacgggctgctggacactgtcgccaggcccgagtccctgcagcaggcccggctgtacgagaacctgcccccggctgcgctacggaagctgctgcacgcggagcccgagcggtaccgccactgctctttcgtgccagagaccttcgagcgggcgtcagccatcccgctggacgatgcctcctcgggccccatccaggtcaggggccgcctggactgtgggatggccttcgccggggatgaggtgctggtgcagctcctttcgggagacaaggcgcccgaggggcggcttcggggccgcgtgctgggcgtgctgaagaggaagaggcacgagctggcgtttgtgtgccgcatggacacgtgggacccgcgcatcatggtccccatcaatggctccgtgaccaagatcttcgtggccgagctgaaggacccatcgcaggtccccatctacagcctccggaagggccggctgcagcgtgtggggcttgagaggctcaccgccgaggcccggcacagccggctcttctgggtccaaattgtcctgtggcggcaaggcttctactacccgctgggcatcgtccgggaggtgctgcctgaggccagcacctgggagcagggcctccgcatcctcggcctggagtacagcttgagggtgcccccgtcggaccaggccaccatcaccaaggtgctgcagaaataccacacggagcttggccgggttgccggccgccgagaggactgccgcgccttcttgaccttcactgtggacccccagggcgcctgcaacctcgatgatgccctcagtgtccgagacctgggtcccaggtgcgaggtggctgtgcacatcactgatgtggccagcttcgtgcccagggacggggtgctggacgtggaggcgcgaaggcagggcgctgcgttctatgcccccggcagggagccagtgcccatgctgccggccagcctctgccaggacgtcctcagcctcctgcctggccgggaccgcctggccatctccctgttcctcaccatggagaaggccagtggccagctgaagagcctgcgctttgcaccctccgtggtccagtctgaccgccagctgtcctacgaggaggcggaggaggtgatcaggcagcacccgggtgccggccgtgagctgccggcccgcctggactccgtggacgcctgcgtcgtggccgcgtgctacttctctcggctgctgcgccggcaccgcctgcggtccgactgcttctatgagcagccggacgaggacggcaccctgggcttccgcgcggcccacatcatggtgaaggagtacatgattcagtttaataggctcgtggctgagttcctggtgggcagcgagtgcacgcggacggtcacgcctctgcggtggcagccagcaccccgcagccagcagctcaaggccctgtgtgagaagcatggggaccgggtgcccctgtcactgcacctcggccaccacctgcacggcggcgggggcagtccccccgacacgcggctgcacctcctggcctccctctggaagcaggtccagtttgctgcccgcacccaggactacgaacagatggtggacttggtcaccacggacgacatgcacccattcctggctcctgcaggccgcgacctccgcaaggccctggagcgctcggcgttcggccgctgcgcccggggccaccagcagcagggcggccactactcgctgcaggtggactggtacacgtgggccacctcgcccatccgcaggtacctggacgtggtgttgcagcggcagatcctgctggcgctgggccatgggggctctgcctactctgccagggacatcgatgggctctgccaggccttcagcctccagcacgcacttgcccagagctatcagcggcgggcgcgcagcctgcacctggccgtgcagctcaaggcccagcctctggacaagctgggcttcgtggtggacgtggaggcgggctcccgctgcttccggctgctcttccccagcaaccgggagacgctgcctgacccctgccccgtcccctacggctccctgcagctggccgagcacccccacgccctggcaggccggccgggcctgcggctcctgtggcggcgccgtgtctactcagcgcagggatccagcccgcccctgccactgcctggcactgtgccggacccacacaccctggccgtggagacggccctgtggaagcagctgctggagctggtggagctgcagcgctggccggaggcggctgctctcatccaggagaagggcgaggcgtcccagcggcgggagctggtgcaggtgcagcggagccactgtggccatttcctggaggtggcccgggagctgggcagtggggacaccctgcaggtgcagctcggcaccagcctgcagcacggcttcctggtaccgagccctcagctctggacggtggcaccgggcttcagcctctgcctggagcacgtggagcggcccggagactgcttctcaggccgtgtgtaccgggccccgagggaccggtaccgcgacgtggatgagtacgcctgcgtgtgggaaccattctgcgccctggagtcggccaccggcgcggttgccgagaatgactccgtcacacttcagcacctgagtgtctcctgggaggcgtcacggacgccgcaggggcagctgcagggcgccttccgcctggaggccgccttcctcgaggagaactgtgccgacatcaacttcagctgctgctacctctgcatccggctcgaggggctgccggctcccacggccagcccacgccctgggcccagcagcctcggccctggcctgaatgttgaccccggcacgtatacctgggtggcccacgggcagacgcaggactgggaccaggagcgccgggcagaccggcaggaggctcccagacgggtgcacctcttcgtccaccacatgggcatggagaaggttccggaagaggtgctgaggccgggcaccctgttcaccgttgagctgctgcccaagcagcttcctgatctccgcaaggaggaagccgtgcgtggactagaggaggcgtccccgctggtcaccagcatcgcactgggccggcctgtcccgcagcccctctgcagagtgatccccagcaggttcctggagcggcagacctacaacatccccggaggccgccacaagctgaaccccagccagaacgtggcggtcagggaggctctggagaagcctttcacggtcattcagggcccaccaggtacagggaagacgatcgtgggcctccacatcgtattctggtttcataaatcaaaccaggagcaggtgcagcccggaggccccccccgtggggagaagcggctggggggtccctgcatcttgtactgcggcccctccaacaagtcggtggatgtcctggcaggactgctcctgagaaggatggagctgaagcccctccgtgtgtacagtgagcaggctgaggccagcgagttcccagtgccgcgtgtgggcagcaggaagctgctcaggaagagcccccgggaggggaggccgaaccagagcctcaggagcatcaccctgcaccaccggatccggcaggcccccaacccttactcgtcggaaatcaaggcctttgacacccggctgcagagaggggagctcttctccagggaggacctggtctggtacaagaaggtcttgtgggaggctcggaagttcgagctggaccggcatgaggtcatcctctgcacctgctcctgtgcagcctctgccagcctcaaaatcctggacgtgaggcagatccttgttgacgaggcaggcatggccacggaacctgaaaccctcatccccctggtgcagttcccacaggccgagaaggtggttcttctcggagaccacaagcagctgcggcctgtggtcaagaatgagcggctgcaaaacctgggtctggaccggtctctgttcgagcggtaccacgaggacgcacatatgctggacactcagtaccgcatgcatgagggcatctgtgccttcccctctgtggcgttctacaagagcaagctgaagacgtggcagggcctgaggaggccgcccagtgtcctgggccacgctggcaaggagagctgccctgtcatctttggccacgtgcagggccacgagcggagcctgctggtgtccacggacgaagggaatgagaactccaaggccaacctggaggaggtggctgaggtggtccgtatcaccaagcagctgaccctggggaggaccgtagagccccaggacatcgccgtcctcacgccctacaacgcgcaggcctctgagatcagcaaggcccttcggcgagagggcatcgccggggtggccgtgtcctccatcaccaagagccaggggagcgagtggcgctatgtgctggtgagcaccgtccgcacctgtgccaagagcgacctggaccagcggcccaccaagagctggctcaagaagtttctgggcttcgttgtggaccccaaccaagtgaatgtggctgtcacgcgggcccaggaggggctctgcctgatcggagaccacctccttctgcgctgctgccccctctggcgtagcctcctggacttctgcgaggctcagcagaccctcgtgcctgccggccaggtgcgcgtctgcaggaggccaactatgccttcctgaagagccctctccacctgcaaggtgccaggactgggagggaaagtccagggcccccccaccctccccaccaccctccgccatcagcctgcctgggccagcctgcctcctggcctcaggatctccctggggcaggaagtgggtggacttggaggaggaacggcaagggccggtccccacactcttgctcctcctgggcatctggggacaggaacacctctcagggtggccaagaggggcttccttgggaccagtgcaaggaagttctgctgtccctgggaagtctcagaaccaggaaggaaaccctgtggaggctgccctttgacctttggacgccacctgtaatcactctggatggtgtctgtggagttcaggtttattttctatttaaatgaactaaaagccagcatcctgccgggcgtggtggctcacgcccataatccagcactttgggaagccgaggcgggcagatcacctgaggtcaggagttcgagaccatcctggccaacatggtgaaaccccgtctccactaaaaatacaaaattagctgggcgtggtggcgggcgcctgtaatcccagctacttgggaggctgaggcaggagaatcgcttgactccaggaggcgcaggctgcagtgagctctgatggtaccactgcactccagcctgggtgacagagcgagaccctgtcttaaaaaaaaaaacaaaacaaaaccagcatctgttactggaacagagacctcagcccaagctcaggacaaggaggcctccctgggagagggggcctttctcccaccatcaccgcctgtgtccttcaggggctgctggagggcagcccaaagccaggggcccaccccgccccaccccaccccaccccaccctctgaaatgtgaaaagcctgcactcttcctgtgggctgcgcagtgggctcgggggttggggggtgccggggcgatgcttccaccctggactgagtgttcggggcagcctgctcggctgtatgtttctcgcacgctcacccgcttcagtgcggctctgtccctgctgccgcctccagtcctgagggaggggcttgccccaccttctacattccaatttttatatctttgaattatgtgattagatattaatttaatgataaaaccactctgaaagctcttctca

(19) United States
(12) Patent Application Publication
(10) Pub. No.: US 2013/0303400 A1
(43) Pub. Date: Nov. 14, 2013

(54) MULTIMARKER PANEL

(71) Applicants: Robert Zeillinger, Blumau-Neurisshof (AT); Dietmar Pils, Wien (AT); Dan Cacsire Castillo Tong, Wien (AT)

(72) Inventors: Robert Zeillinger, Blumau-Neurisshof (AT); Dietmar Pils, Wien (AT); Dan Cacsire Castillo Tong, Wien (AT)

(21) Appl. No.: 13/989,733

(22) PCT Filed: Nov. 28, 2011
(86) PCT No.: PCT/EP2011/071162

§ 371 Date: Jul. 3, 2013

(30) Foreign Application Priority Data
Nov. 26, 2010 (EP) 10192692.1
Mar. 22, 2011 (EP) 11159273.9

(51) Int. Cl. C12Q 1/68

(52) U.S. Cl. CPC C12Q 1/6886
USPC 506/9; 506/16

(57) ABSTRACT

The invention is directed to a method of diagnosing a malignant ovarian tumor disease in a subject, which comprises —providing a sample of peripheral blood cells (PBC) of the subject, —measuring the expression of a multimarker gene panel comprising at least NEAT1, BC037918, C1 orf63, PRIC285, OSM, and optionally further genes or protein markers, and —comparing to a reference value, the differential expression being indicative of a malignant ovarian tumor, and a set of reagents to determine the expression of such a multimarker panel, as well as the use of a PBC-expression based test to improve the diagnosis of ovarian cancer. The invention further relates to a method of determining the expression of at least one of the RPL21, RPL9 and/or SH3BGRL3 genes in a PBC sample of a subject as internal control.

### BACKGROUND

A tumor marker, also called marker or biomarker, is a substance sometimes found in an increased or decreased amount in the blood, other body fluids, or tissues and which may mean that a certain type of cancer is in the body. There are many different tumor markers, each indicative of a particular disease process, and they are used in oncology as a diagnostic or prognostic marker or used to monitor cancer therapy.

Usually, tumor-specific markers are overexpressed in tumor tissue compared to normal tissue and therefore determined in tumor specimens. Thus, the expression of tumour-specific genes in cancerous tissue is sometimes investigated to gain information about prognostic markers and molecular targets for diagnosis or chemical and/or immunological therapy.

One of the most deadly malignant diseases in women is ovarian cancer. The high risk of dying is probably due to late diagnosis, i.e. 67% of patients are diagnosed with advanced disease, 15 percentage points more than in pancreatic cancer, known to be diagnosed frequently at advanced stages. Approximately 14,600 deaths (5% of all cancer types) were estimated in 2009 only for the United States of America, rendering this type of tumor to the fifth frequent cause of cancer death in females. The five-year overall survival (OS) rate is only 46% among all stages. Patients with stage I disease have a five-year OS rate of more than 90%, whereas patients with advanced disease less than 30%. One reason for the low five-year OS rate is the fact that ovarian cancer presents with few, if any, specific symptoms. Therefore markers for early detection of ovarian cancer could improve OS. Up to now no screening markers are recommended or routinely used for early detection of ovarian cancer.

Only a fraction of ovarian tumors are malignant. Being able to determine the malignant ones preoperatively and without employing biopsies, would permit a better preoperative management and improved treatment options.

One of the known serum marker for ovarian cancer is CA-125, described for the first time in 1981 as a murine monoclonal antibody (OC125) reacting against ovarian cancer cell lines and cryopreserved ovarian cancer tissues but not with benign tissues or other carcinomas. CA-125 is a coelomic epithelial antigen produced by mesothelial cells in the peritoneum, pleural cavity and pericardium. Serum CA-125 levels are measurably increased in about 80% of patients with ovarian cancer, to a lesser extent in patients with early stages. Sensitivity of CA-125 is lower than 60% in early stages. But serum concentrations can be elevated by a number of common benign gynecologic conditions, including endometriosis, leiomyomas, congestive heart failure and liver cirrhosis. In addition, serum concentrations of CA-125 are higher in premenopausal women, which have low prevalence of ovarian cancer, compared to postmenopausal women. These facts all together results in an impaired sensitivity and specificity for CA-125. Nevertheless, there are many papers dealing with CA-125 as marker for early detection, diagnosis, response prediction and monitoring, disease recurrence, and for distinguishing malignant from benign pelvic tumors; CA-125 is even discussed as a therapeutic target.

One of the possibilities to increase the sensitivity and specificity of CA-125 is to expand this single marker to a marker panel, i.e. add other serum markers and build a statistical model to detect ovarian cancer.

In 2004 Zhang et al. published a four marker panel comprised of CA-125 and three by mass spectroscopy (SELDI) newly identified serum protein peaks, identified as apolipoprotein A1 (down-regulated in malignant tumors), a truncated form of transthyretin (down-regulated), and a cleaved fragment of inter-α-trypsin inhibitor heavy chain H4 (up-regulated) (Zhang Z et al. Cancer Res 2004; 64: 5882-90). A multivariate model combining the three biomarkers and CA-125 reached a sensitivity of 74% by a fixed specificity of 97% for detection of early stage epithelial ovarian cancer (EOC). This set of biomarkers was amended by four further serum protein peaks leading to a commercialized FDA cleared blood test for assessment of the likelihood that an ovarian mass is malignant before it is removed, called OVA1™ (Quest Diagnostics, Madison, N.J., USA).

Mor et al. described in 2005 four new serum markers, Leptin, Prolactin, OPN, IGF-II, found by a rolling circle amplification (RCA) immunoassay microarray approach, which showed an overall sensitivity and specificity of approx. 95%, including 19% early stage patients in a combined predictive model (Mor G et al. Proc Natl Acad Sci USA 2005; 102: 7677-82). Later on, CA-125 and MIF were added to this four-marker-panel, which increased the specificity to 99.4% by a sensitivity of 95.3%. However, with this marker panel, 11.1% stages I and II samples (4 of 36) were misclassified (Visintin I et al. Clin Cancer Res 2008; 14: 1065-72).

Recently, Yurkovetsky et al. described a four serum marker panel, HE4, CEA, VCAM-1, and CA-125, for early detection of ovarian cancer. A model derived from these four serum markers provided a diagnostic power of 86% sensitivity for early stage and 93% sensitivity for late stage ovarian cancer at 98% specificity (Yurkovetsky Z et al. J Clin Oncol 2010; 28: 2159-66).

Häusler et al. (British Journal of Cancer (2010) 103, 693-700) describe a characteristic miRNA fingerprint in peripheral blood of ovarian cancer patients employing a cellular fraction, referring to circular tumor cells, stromal and myeloid progenitors and hematopoietic cells with a low predictive power.

US2010/0216137 A1 discloses a method of gene expression profiling for identification, monitoring and treatment of avarian cancer, employing a sample providing a source of RNAs. Such a sample may be whole blood or a population of cells or tissue from a subject.

Another approach to find prognostic markers for early detection of ovarian cancer would be to use a different component from blood, namely peripheral blood cells (PBC) instead of serum. In breast cancer the gene expression patterns from PBC were used for early detection of breast cancer, however with low selectivity. A set of 37 genes was identified which expression in peripheral blood cells could detect in at least 82% of breast cancer patients (Sharma P et al. Breast Cancer Res 2005; 7: R634-44). A set of 738 genes was identified which discriminated breast cancer patients from controls without malignant breast disease with an estimated prediction accuracy of only 79.5% (80.6% sensitivity and 78.3% specificity) (Aaroe J et al. Breast Cancer Res 2010; 12: R7).

Although expression profile analysis of tissue biopsies have identified the presence of tumor-associated transcriptomes that may be used to diagnose cancer and to distinguish between malignant or benign disease, the required biopsy of tissue makes such methods of diagnosis unattractive. Compared to tissue biopsies, cells in the peripheral blood, in particular circulating PBCs, are much more accessible.

There remains a need in the art for new and effective tools to facilitate early diagnosis of ovarian cancer that could more accurately diagnose malignant ovarian tumor disease in patients from other non-malignant diseases and would reduce unnecessary diagnostic surgery and biopsies.

The object of the present invention was to find new prognostic biomarkers to diagnose malignant ovarian tumor disease in a subject.

The object is achieved by the provision of the embodiments of the present invention.

### SUMMARY OF THE INVENTION

The present invention refers to a method of diagnosing a malignant ovarian tumor disease in a subject, which comprises
providing a sample of peripheral blood cells (PBC) of the subject,
measuring the expression of a multimarker gene panel comprising at least NEAT1, BC037918, C1orf63, PRIC285, OSM, and optionally further gene or protein markers,

and
comparing to a reference value, e.g. derived from non-cancerous patients or healthy controls,

the differential expression being indicative of a malignant ovarian tumor.

According to a specific aspect, the sample is obtained from a blood fraction enriched in white blood cells, including granulocytes and optionally lymphocytes. Preferably the PBC fraction is used, wherein as white blood cells a majority of granulocytes is contained. An exemplary blood fraction is obtained by density fractionation, such as buoyant one or more-step gradient procedures to obtain a fraction with a high density, which contains the granulocytes and lymphocytes, e.g. as obtained by a sample preparation method as described in Brandt et a. (Clinical Chemistry 42(11), 1881-1882 (1996), or alternative methods to obtain a PBC fraction, which is a white blood cell fraction containing granulocytes or a fraction wherein a majority of the white blood cells are granulocytes. Besides, other white blood cells which are not granulocytes (agranulocytes) may be contained, e.g. lymphocytes and monocytes. Also PBMC may be contained.

Such PBC fraction is preferred that is obtained upon reducing the content of potentially present epithelial cells, including circulating tumor cells, which includes (partial or quantitative) removal or depletion of epithelial cells.

The preferred method employs a sample, which is obtained from a fraction containing blood cells, in particular peripheral white blood cells, e.g. a blood sample, optionally depleted from epithelial cells and/or enriched in granulocytes or lymphocytes.

Specifically, the expression of at least one of the further genes B4GALT1, CCR2, CFP, ZNF419, PAPOLG, NOXA1, DIS3 and/or AP2A1 is determined.

According to a specific embodiment the expression of the markers NEAT1, BC037918, C1orf63, PRIC285, OSM, DIS3 and CCR2, or at least these markers is determined, possibly in combination with further genes markers and/or protein markers.

According to another specific embodiment, the expression of at least NEAT1, BC037918, C1orf63, PRIC285, OSM, B4GALT1, CCR2, CFP, ZNF419, PAPOLG, NOXA1, DIS3 and AP2A1 is determined.

In a particularly preferred embodiment the expression of at least one of the further house-keeping genes RPL21, RPL9 and/or SH3BGRL3 is determined as internal control, preferably at least two or three of these internal control genes. Any other house-keeping gene may be used as internal control for the purpose of the invention.

Preferably the nucleic acid and/or protein expression is determined. According to a specific embodiment, the nucleic acid expression is determined, which is indicative for the gene expression, preferably the mRNA. According to a specific example, the mRNA of the individual genes or markers in the PBC sample, as determined either quantitatively or qualitatively, e.g. the differential expression, such as a differential expression, e.g. an increased or decreased expression, is indicative of malignant ovarian tumor disease, such as ovarian cancer.

According to a specific aspect the method can be combined with any other diagnostic method for determining ovarian cancer, specifically another diagnostic method selected from a screening method for serum proteins or proteins from other bodily fluids, which are indicative of ovarian cancer.

According to a specific embodiment, the nucleic acid or protein expression of the serum or plasma proteins or proteins from other bodily fluids is determined.

According to another specific embodiment, the method can be combined with another diagnostic method selected from determination methods for clinical parameters, which are indicative of ovarian cancer, such as transvaginal ultrasound. Specific combinations of methods according to the invention encompass e.g.
a method for determining the nucleic acid expression, such as mRNA, of genes in the PBC sample according to the invention, e.g. the multimarker panel according to the invention,
and
a method for determining specific serum or plasma proteins in bodily fluids, such as serum or blood, indicative of malignant ovarian tumor disease, e.g. by their protein expression, which always encompasses the determination of the proteins as such or their binding or other functional properties,
and/or
a method for determining clinical paramerters, indicative of malignant ovarian tumor disease, such as transvaginal ultrasound.

Such combinations may contribute to improve the diagnosis of malignant disease.

Specifically the serum proteins are selected from the group consisting of CA-125, HE4, CEA, VCAM-1, MIF, Leptin, Prolactin, OPN (osteopondin), IGF-II (IGF2), apolipoprotein A1, transthyretin or truncated transthyretin, ITIH4, hepcidin, R2-microglobulin, transferrin, CTAP3 and inter-alpha-trypsin inhibitor heavy chain H4, or a cleavage fragment thereof.

According to a specific embodiment, at least NEAT1, BC037918, C1orf63, PRIC285, OSM, and further at least MIF, Prolactin, CA125, Leptin and IGF-II are determined. Thus, in a preferred embodiment the gene markers NEAT1, BC037918, C1orf63, PRIC285 and OSM are determined in combination with the protein markers MIF, Prolactin, CA125, Leptin and IGF-II, possibly combined with further gene markers and/or protein markers.

According to another specific embodiment, at least NEAT1, BC037918, C1orf63, PRIC285, OSM, DIS3, CCR2, and further at least MIF, Prolactin, CA125, Leptin and IGF-II are determined. Thus, in a preferred embodiment the gene markers NEAT1, BC037918, C1orf63, PRIC285, OSM, DIS3 and CCR2 are determined in combination with the protein markers MIF, Prolactin, CA125, Leptin and IGF-II, possibly combined with further gene markers and/or protein markers.

According to another specific embodiment, at least NEAT1, BC037918, C1orf63, PRIC285, OSM, B4GALT1, CCR2, CFP, ZNF419, PAPOLG, NOXA1, DIS3, AP2A1 and further at least MIF, Prolactin, CA125, Leptin, IGF-II and OPN are determined. Thus, in another preferred embodiment the gene markers NEAT1, BC037918, C1orf63, PRIC285, OSM, B4GALT1, CCR2, CFP, ZNF419, PAPOLG, NOXA1, DIS3, AP2A1 are determined in combination with the protein markers MIF, Prolactin, CA125, Leptin, IGF-II and OPN, possibly combined with further gene markers and/or protein markers.

Specifically the expression is determined qualitatively and/or quantitatively, e.g. by Northern blot or other hybridization based methods, RT-qPCR, microarrays, ELISA, EIA, RIA, western blot, protein arrays, immunocytochemistry or immunohistochemistry methods. Other methods of determining the expression of genes or non-coding sequences associated therewith, including any of corresponding RNA, such as mRNA, or DNA specimen, or else proteins, peptides, precursors or ligands are feasible. Such expression or the expression level may be indicative of a malignant ovarian tumor. In particular the differential expression levels as compared to a reference may be determined.

According to a specific aspect the method provides for the prediction of malignant ovarian tumor even at an early stage, in particular of the FIGO I and/or II (I/II) stage, with a specificity of at least 99%, preferably with a sensitivity of at least 70%, specifically at least 74%, more specifically with a sensitivity of at least 80%, at least 85%, at least 90% or even more than 95%. In preferred cases a sensitivity of 95.5-99% at a specificity of at least 99.5% can be reached.

The preferred method differentiates between a malignant ovarian tumor and a benign (e.g. ovarian cyst or cystadenoma) or low malignant potential (LMP) tumor (e.g. a borderline tumor). A benign tumor as understood herein is a tumor that lacks the ability to metastasize.

According to another aspect the present invention provides for a set of reagents to determine the expression of a multimarker panel in a PBC fraction, which is at least consisting of NEAT1, BC037918, C1orf63, PRIC285, OSM, and optionally at least one of B4GALT1, CCR2, CFP, ZNF419, PAPOLG, NOXA1, DIS3 and/or AP2A1, which is herein understood as the PBC multimarker panel. The set according to the invention specifically comprises reagents to determine the individual markers of said panel. Specifically reagents are comprised to determine mRNA of the individual markers.

The set according to the invention specifically may contain reagents for a limited number of individual markers of the PBC multimarker panel only. Preferably such multimarker panel comprises less than 100 individual markers, more preferred less than 50 individual markers, even more preferred less than 40, or less than 30 or less than 20, or less than 10 individual markers. According to a specific embodiment, the set comprises only those reagents of the PBC multimarker panel as necessary to determine a limited or the above specified individual markers only, specifically only those reagents as necessary to determine the PBC multimarker panel consisting of NEAT1, BC037918, C1orf63, PRIC285, OSM, and optionally at least one of B4GALT1, CCR2, CFP, ZNF419, PAPOLG, NOXA1, DIS3 and/or AP2A1, optionally in combination with reagents to determine serum markers.

According to a specific embodiment, the set according to the invention further comprises means to prepare a PBC fraction. Such means include buffer or other auxiliary reagents or tools to enrich and/or fractionate white blood cells, cell lysis reagents, internal controls, negative controls, etc.

The preferred set further contains reagents for determining the expression of at least one of the RPL21, RPL9, and/or SH3BGRL3 genes as internal control.

Preferably the set comprises primers, optionally together with probes, to perform the RT-qPCR analysis of the individual markers, optionally together with further tools to perform such analysis.

It is preferred that the set comprises probes to specifically hybridise with gene transcription products of the individual markers.

According to a specific aspect the invention refers to the use of the set in a method of diagnosing a malignant ovarian tumor disease in a subject, such as a method employing the PBC multimarker panel according to the invention, or a method according to the invention as described herein.

According to another aspect the invention provides for the use of a PBC-expression based test or method to improve a serum protein based test for diagnosing ovarian cancer, preferably a test to determine mRNA in a PBC fraction.

Specifically the PBC-expression based test is a method according to the invention or any other method employing the PBC multimarker panel according to the invention.

Still, according to another aspect the invention provides for a method of determining the expression of at least one of the RPL21, RPL9 and/or SH3BGRL3 genes, preferably at least two or three of them, in a PBC fraction such as used in the method according to the invention, of a subject, e.g. a human subject at risk of a disease, such as a malignant ovarian tumor disease like cancer, as normalization markers or internal control to quantify the expression of further genes other than the internal control genes, such as those which are indicative of a disorder or a disease by the respective overexpression/upregulation or downregulation, e.g. to indicate or diagnose a disorder or disease, in particular malignant ovarian tumor disease like cancer.

### FIGURES

FIG. 1: Nucleotide sequence of BC037918 (SEQ ID 1): cDNA sequence information, human.

FIG. 2: Nucleotide sequence of PRIC285 (SEQ ID 2): cDNA sequence information, transcript variant 1, including other variants possibly used, human.

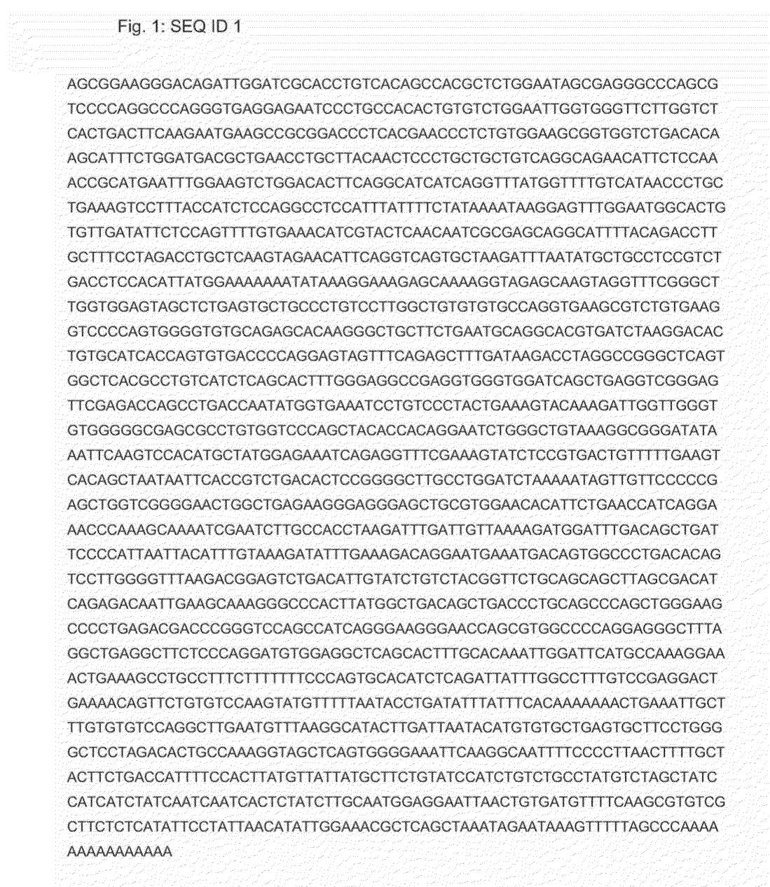

### DETAILED DESCRIPTION

The invention is directed to the preparation and analysis of expression profiles of peripheral blood cells, such as obtained from peripheral white blood cells or a PBC fraction enriched in white blood cells, specifically in samples from human subjects, isolated from subjects at risk of (malignant) ovarian tumor disease, and the identification of PBC transcriptional gene signatures capable of distinguishing between subjects suffering from a malignant or benign tumor.

By the inventive gene signature derived from the expression in PBCs early diagnosis of ovarian cancer is possible according to the invention with unexpected high specificity and sensitivity. LMP tumors and cystadenomas can even be diagnosed as benign tumors.

The present invention is directed to the utilization of at least one “transcriptional gene signature”, also referred to herein as a “gene signature,” “expression signature,” “transcriptome,” “profile,” or “gene profile”, of PBCs, in particular of a PBC fraction comprising white blood cells, i.e., PBC-associated, and in particular granulocytes and optionally lymphocytes associated, transcriptional gene signatures, to determine whether a subject is suffering from a malignant ovarian tumor disease. The present invention is also directed to the use of PBC-associated transcriptomes for the optional determination of whether a subject with an ovarian tumor is suffering from a malignancy or not. The present invention is based on the finding of PBC-associated and ovarian cancer-associated markers, specifically transcriptomes. The presence or the level of such markers as determined according to the invention specifically is indicative of malignant ovarian tumors. In particular, the invention is based on the identification of PBC- and ovarian cancer-associated biomarkers, which may be grouped in a multimarker panel based on their utility in the diagnosis, prognosis, monitoring, and/or treatment of malignant ovarian tumor disease.

As used herein, the term “biomarker” or “marker” includes a polynucleotide (e.g., gene, transcript, expressed sequence tag (EST), etc.) or polypeptide molecule that is substantially modulated (i.e., upregulated or downregulated) in quantity in peripheral blood cells of subjects, specifically human beings, with malignant ovarian tumor disease, including ovarian cancer of early or late stage, with high, medium or low metastatic potential as compared to a subject substantially free of such tumor (e.g. a healthy subject) or a subject suffering from a benign tumor. In certain embodiments, the PBC-associated biomarkers of the invention include the polynucleotides, their corresponding gene products, expression products and fragments, variants, such as splice variants or other functionally equivalent variants, homologs and isoforms thereof. Specifically the individual markers of the multimarker panel are determined by preparing samples of mRNAs in the PBC sample or fraction, and determining the presence and/or level of the mRNAs.

The term “inventive biomarkers” particularly refers to the individual markers of a panel that comprises at least NEAT1, BC037918, C1orf63, PRIC285, OSM, and optionally further genes or protein markers, such as at least one of the further genes B4GALT1, CCR2, CFP, ZNF419, PAPOLG, NOXA1, DIS3 and/or AP2A1, which panel is herein also called multimarker panel or PBC multimarker panel. The term shall also refer to the panel consisting of the genes NEAT1, BC037918, C1orf63, PRIC285, OSM, or alternatively consisting of the genes NEAT1, BC037918, C1orf63, PRIC285, OSM, DIS3 and CCR2, or alternatively consisting of the genes NEAT1, BC037918, C1orf63, PRIC285, OSM, B4GALT1, CCR2, CFP, ZNF419, PAPOLG, NOXA1, DIS3 and AP2A1, alone or in combination with a panel of protein markers, such as a panel including at least one of CA-125, HE4, CEA, VCAM-1, MIF, Leptin, Prolactin, OPN (osteopondin), IGF-II (IGF2), apolipoprotein A1, transthyretin or truncated transthyretin, ITIH4, hepcidin, R2-microglobulin, transferrin, CTAP3 and inter-alpha-trypsin inhibitor heavy chain H4, or a cleavage fragment thereof. A preferred panel is panel with a limited number of different biomarkers, which e.g. consists of 5-15, such as 5, 6 or 7 individual gene markers, and 1-10, such as 3, 4, 5 or 6 individual protein markers of these biomarkers.

The biomarkes of the invention are specifically of human origin and provided in Table 1 including the UniGene accession numbers, gene symbols and HUGO nomenclature, where applicable, and the directions of their modulation (i.e. upregulation or downregulation).

TABLE 1
PBC-associated biomarkers indicative of an ovarian malignancy

| | | | | UniGene | |
|---|---|---|---|---|---|
| | | | | acc. no. | |
| Probe | Gene | | | (Build | |
| ID^{b} | symbol^{c} | Gene name | Function | #226) | Regulation^{a} |
| 115368 | AP2A1 | adaptor-related protein | Clathrin coat | Hs.467125 | Down |
| | | complex 2, alpha 1 subunit | assembly | | |
| 142487 | B4GALT1 | UDP-Gal:betaGlcNAc beta | Galactosyltransferase | Hs.272011 | No |
| | | 1,4-galactosyltransferase, | | | |
| | | polypept. 1 | | | |
| 157342 | C1orf63 | chromosome 1 ORF 63 | Unknown | Hs.259412 | Down |
| 110071 | CCR2 | chemokine (C-C motif) | Chemokine receptor | Hs.511794 | Up |
| | | receptor 2 | | | |
| 119290 | CFP | complement factor properdin | Alternative pathway | Hs.53155 | Down |
| | | | for complement | | |
| | | | activation | | |
| 105743 | DIS3 | DIS3 mitotic control homolog | RNase, part of the | Hs.726220 | No |
| | | (S. cerevisiae) | exosome complex | | |
| 228089 | NEAT1 | non-protein coding RNA 84 | Non-coding RNA | Hs.523789 | Up |
| 182018 | NOXA1 | NADPH oxidase activator 1 | Activates NADPH | Hs.495554 | Down |
| | | | oxidases | | |
| 205406 | OSM | oncostatin M | IL-6 family cytokine | Hs.248156 | Down |
| 161567 | PAPOLG | poly(A) polymerase gamma | Poly(A) polymerase | Hs.387471 | Down |
| 162222 | PRIC285^{d} | peroxisomal proliferator- | Nuclear | Hs.517180 | Down |
| | | activated receptor A | transcriptional | | |
| | | interacting complex 285 | coactivator for | | |
| | | | several nuclear | | |
| | | | receptors | | |
| 109227 | ZNF419 | zinc finger protein 419 | Zinc finger protein | Hs.467358 | No |
| 713562 | N/A^{e} | (no ORF in transcript | Non-coding RNA | Hs.212226 | Up |
| | | BC037918) | | | |

^{a}Significant down- or up-regulation in PBCs of EOC patients compared to healthy blood donors.
^{b}ProbeIDs as used in the examples below.
^{c}HUGO nomenclature, where applicable.
^{d}see SEQ ID 2.
^{e}see SEQ ID 1.

The biomarker panel according to the invention may be combined with further tests, e.g. to determine further PBC markers or markers of white blood cells, such as granulocyte or lymphocyte markers, or serum markers. Preferred marker combinations are with those known to relate to ovarian cancer or more general to epithelial markers that are indicative of carcinomas. Any marker combination of at least the inventive genes and optionally one or more markers associated with cancer, which increases the selectivity and/or the sensitivity is considered a preferred combination to determine the risk of ovarian tumor disease.

Preferably further biomarkers that are not associated with the disease, but stably expressed, so-called house-keeping genes such as PBC house-keeping genes, are determined as internal control, e.g. simultaneously, in parallel or consecutively within the same sample or the same type of sample, to obtain reference values for normalization of the expression levels to facilitate the comparison of test results. The normalized level of gene expression in a test sample is then typically compared with the reference (external control) value that is usually obtained from healthy subjects or subjects with a benign tumor.

The biomarkers of the invention may be polynucleotides, their corresponding gene products, and fragments, homologs and isoforms thereof, that are substantially modulated (i.e. upregulated or downregulated) in PBCs of patients with malignant ovarian tumor disease compared to PBCs of subjects substantially free of such tumor and/or in PBCs of patients with a benign tumor. Any differential expression of such biomarkers, e.g. by upregulating or downregulating as compared to a reference, such as obtained from healthy subjects or subjects suffering from a benign tumor, might indicate a higher or lower risk of a malignant ovarian tumor disease.

As used herein, “a set of biomarkers” or a “panel of biomarkers” also called “multimarker panel” includes a set of polynucleotides, their corresponding gene products, including transcripts and proteins, and fragments, homologs and isoforms thereof that may be used to distinguish patients with malignant ovarian tumor disease from subjects substantially free of such tumor and/or patients with a benign tumor.

Preferably, for the purposes of the present invention, expression levels of the substantially modulated, i.e., upregulated or downregulated, PBC-associated biomarkers of the invention are respectively increased or decreased by an abnormal magnitude, wherein the level of expression is aberrant, e.g. outside the standard deviation for the same PBC-associated biomarker in PBCs from healthy subjects or from subjects suffering from a benign tumor. Most preferably, the substantially modulated PBC-associated biomarker is upregulated or downregulated relative to a reference value by at least an aberrant 1.5-, 2-, 3-, or 4-fold change or more. It is not necessary, that all of these markers alone are significantly up- or downregulated in each cancer sample. A risk-score, calculated with the coefficients provided in Table 3 (LASSO model), is preferably used for indicating the malignant disease.

The term “PBC” as used herein shall always refer to peripheral blood cells as found in samples of blood or other body fluids, in particular of human subjects, or immobilized on surfaces, in particular white blood cells, such as granulocytes or lymphocytes, but also peripheral blood mononuclear cells (PBMCs), e.g. obtained in a blood or other body fluid or fraction of such body fluid containing epithelial and/or mononuclear cells, preferably depleted from such epithelial cells or disseminated tumor cells and/or enriched in the white blood cells. It is feasible that such a PBC sample as used according to the invention is obtained from extracts of body fluids or tissues, which contain PBCs.

PBC samples are usually taken from peripheral blood using cell separation techniques according to cell harvesting protocols, and are used either freshly prepared or in the frozen state. According to a typical protocol for PBC fraction, wherein a majority of white blood cells are mainly comprising granulocytes and lymphocytes, whole blood is collected by standardized venipuncture with anticoagulants and further processed. Briefly, blood samples are homogenized and processed by density-gradient centrifugation. Then a cell (RNA) lysis buffer is added to the isolated PBC fraction. The PBC lysates are used to extract total RNA immediately or after storage in the frozen state. The mRNA may then be tested according to standard protocols employing commercially available analytical tools with a specificity to determine the individual biomarkers (e.g. RT-qPCR, microarray technology, Next Generation Sequencing).

AP2A1, Adaptor-Related Protein Complex 2, Alpha 1 Subunit

This gene (UniProtKB: 095782) encodes the alpha 1 adaptin subunit of the adaptor protein 2 (AP2 adaptors) complex found in clathrin coated vesicles. The AP-2 complex is a heterotetramer consisting of two large adaptins (alpha or beta), a medium adaptin (mu), and a small adaptin (sigma). The complex is part of the protein coat on the cytoplasmic face of coated vesicles which links clathrin to receptors in vesicles. Alternative splicing of this gene results in two transcript variants encoding two different isoforms. Adaptor protein complexes function in protein transport via transport vesicles in different membrane traffic pathways. Adaptor protein complexes are vesicle coat components and appear to be involved in cargo selection and vesicle formation.

B4GALT1, UDP-Gal:betaGlcNAc beta 1,4-galactosyltransferase, polypeptide 1

Beta-1,4-galactosyltransferase 1 is an enzyme that in humans is encoded by the B4GALT1 gene (UniProtKB: P15291), which belongs to the glycosyltransferase 7 family. This gene is one of seven beta-1,4-galactosyltransferase (beta4GalT) genes. They encode type II membrane-bound glycoproteins that appear to have exclusive specificity for the donor substrate UDP-galactose; all transfer galactose in a beta1,4 linkage to similar acceptor sugars: GlcNAc, Glc, and Xyl. Each beta4GalT has a distinct function in the biosynthesis of different glycoconjugates and saccharide structures. As type II membrane proteins, they have an N-terminal hydrophobic signal sequence that directs the protein to the Golgi apparatus and which then remains uncleaved to function as a transmembrane anchor. By sequence similarity, the beta4GalTs form four groups: beta4GalT1 and beta4GalT2, beta4GalT3 and beta4GalT4, beta4GalT5 and beta4GalT6, and beta4GalT7. This gene is unique among the beta4GalT genes because it encodes an enzyme that participates both in glycoconjugate and lactose biosynthesis.

C1orf63, Chromosome 1 ORF 63

C1orf63 (Gene ID: 57035) corresponds to a protein coding transcript, whose gene product (hypothetical protein L0057035) has an unknown function and has no homology to any other known protein.

CCR2, Chemokine (C—C motif) Receptor 2

CCR2, short for chemokine (C—C motif) receptor 2, is a chemokine receptor. CCR2 has also recently been designated CD192 (cluster of differentiation 192). This gene (UniProtKB: P41597) encodes two isoforms of a receptor for monocyte chemoattractant protein-1, a chemokine which specifically mediates monocyte chemotaxis. Monocyte chemoattractant protein-1 is involved in monocyte infiltration in inflammatory diseases such as rheumatoid arthritis as well as in the inflammatory response against tumors. The receptors encoded by this gene mediate agonist-dependent calcium mobilization and inhibition of adenylyl cyclase. This gene is located in the chemokine receptor gene cluster region. Two alternatively spliced transcript variants are expressed by the gene.

CFP, Complement Factor Properdin

This gene (Gene ID: 5199) encodes a plasma glycoprotein that positively regulates the alternative complement pathway of the innate immune system. This protein binds to many microbial surfaces and apoptotic cells and stabilizes the C3- and C5-convertase enzyme complexes in a feedback loop that ultimately leads to formation of the membrane attack complex and lysis of the target cell. Mutations in this gene result in two forms of properdin deficiency, which results in high susceptibility to meningococcal infections. Multiple alternatively spliced variants, encoding the same protein, have been identified.

DIS3, DIS3 Mitotic Control Homolog (S. Cerevisiae)

Exosome complex exonuclease RRP44 (Ribosomal RNA-processing protein 44) or Dis3 is an enzyme that in humans is encoded by the DIS3 gene (UniProtKB: Q9Y2L1). Its protein product is an RNase enzyme homologous to the yeast protein Rrp44, and can be part of the exosome complex in the nucleus of eukaryotic cells.

NEAT1, Non-Protein Coding RNA 84 (Gene ID: 283131)

NEAT1 refers to Nuclear Enriched Abundant Transcript 1, a non-protein coding transcript

NOXA1, NADPH Oxidase Activator 1 (Gene ID: 10811)

NOXA1 (UniProtKB: Q86UR1)

NADPH oxidases (NOXs) catalyze the transfer of electrons from NADPH to molecular oxygen to generate reactive oxygen species (ROS). The NOX activator NOXA1 can stimulate both NOX1 (MIM 300225) and NOX2 (CYBB; MIM 300481), but it appears to be more effective in activating NOX1.

OSM, Oncostatin M

Oncostatin M, also known as OSM, is a protein that in humans is encoded by the OSM gene (UniProtKB: P13725). OSM is a pleiotropic cytokine that belongs to the interleukin 6 group of cytokines. Of these cytokines it most closely resembles leukemia inhibitory factor (LIF) in both structure and function. However, it is as yet poorly defined and is proving important in liver development, haematopoeisis, inflammation and possibly CNS development. It is also associated with bone formation and destruction.

PAPOLG, Poly(A) Polymerase Gamma

Poly(A) polymerase gamma is an enzyme that in humans is encoded by the PAPOLG gene (UniProtKB: Q9BWT3). This gene encodes a member of the poly(A) polymerase family which catalyzes template-independent extension of the 3′ end of a DNA/RNA strand. This enzyme shares 60% identity to the well characterized poly(A) polymerase II (PAPII) at the amino acid level. These two enzymes have similar organization of structural and functional domains. This enzyme is exclusively localized in the nucleus and exhibits nonspecific and CPSF (cleavage and polyadenylation specificity factor)/AAUAAA-dependent polyadenylation activity.

PRIC285, Peroxisomal Proliferator-Activated Receptor a Interacting Complex 285, Synonyms: KIAA1769, PRIC COMPLEX 285-KD SUBUNIT, PPAR-Gamma DNA-Binding Domain-Interacting Protein 1 (PDIP1), PDIP1-ALPHA, PDIP1-BETA; (see SEQ ID 2 Providing mRNA Sequence Information, Transcript Variant 1, Including Other Variants Possibly Used, Human)

Peroxisomal proliferator-activated receptor A interacting complex 285, also known as PRIC285, is a human gene (UniProtKB: Q9BYK8). The protein encoded by this gene is a nuclear transcriptional co-activator for peroxisome proliferator activated receptor alpha. The encoded protein contains a zinc finger and is a helicase that appears to be part of the peroxisome proliferator activated receptor alpha interacting complex. This gene is a member of the DNA2/NAM7 helicase gene family.

ZNF419, Zinc Finger Protein 419 (Gene ID: 79744)

ZNF419 codes for a Zn-finger protein (UniProtKB: Q59FT8), propably involved in gene regulation.

BC037918 (No ORF in Transcript BC037918, See SEQ ID 1 Providing cDNA Sequence Information, Human)

Transcript BC037918 has no open reading frame (ORF) and seems to be an untranslated transcript.

RPL21, Ribosomal Protein L21 (Gene ID: 6144)

60S ribosomal protein L21 is a protein that in humans is encoded by the RPL21 gene. Ribosomes, the organelles that catalyze protein synthesis, consist of a small 40S subunit and a large 60S subunit. Together these subunits are composed of 4 RNA species and approximately 80 structurally distinct proteins. This gene encodes a ribosomal protein that is a component of the 60S subunit. The protein belongs to the L21E family of ribosomal proteins. It is located in the cytoplasm.

RPL9, Ribosomal Protein L9 Gene ID: 6133

This gene encodes a ribosomal protein that is a component of the 60S subunit. The protein belongs to the L6P family of ribosomal proteins. It is located in the cytoplasm. As is typical for genes encoding ribosomal proteins, there are multiple processed pseudogenes of this gene dispersed through the genome. Two alternatively spliced transcript variants encoding the same protein have been found for this gene.

SH3BGRL3, SH3 Domain-Binding Glutamic Acid-Rich-Like Protein 3 (Gene ID: 83442)

SH3BGL3 (UniProtKB: SH3L3_HUMAN, Q9H299) could act as a modulator of glutaredoxin biological activity.

The term “subject” as used herein shall refer to any mammal, in particular a human, but also selected from animals, such as those used for tumor models and other animal studies. Preferably the subject shall be human beings who are patients at risk of developing a tumor. Thus, the inventive biomarkers are preferably human biomarkers. The term “patient” as used herein always includes healthy subjects.

The term “malignant ovarian tumor disease” as used herein shall refer to any pathologic condition resulting from a malignant ovarian tumor, such as cancer, including the various stages FIGO I, II, III and IV, a proliferating disease, metastasis, and refractory or recurrent disease. The full FIGO—International Federation of Gynecology and Obstetrics—ovarian cancer surgical staging system is based on Roman numerals as well as letters to designate sub-stages. In general, prognosis depends more upon the main stage. However, the sub-stages can also be important in deciding between treatment recommendations.

Stage I—the Cancer is Limited to the Ovaries

IA—Limited to one ovary and the outer ovarian capsule is not ruptured. There is no tumor on the external surface of the ovary and there is no ascites and/or the washings are negative.

IB—Cancer is present in both ovaries, but the outer capsule is intact and there is no tumor on external surface. There is no ascites and the washings are negative.

IC—The cancer is either Stage IA or IB level but the capsule is ruptured or there is tumor on the ovarian surface or malignant cells are present in ascites or washings.

Stage I—Cancer involves one or both ovaries with spread to other pelvic organs or surfaces.

IIA—Extension or implants onto the uterus and/or fallopian tube. The washings are negative washings and there is no ascites.

IIB—Extension or implants onto other pelvic tissues. The washings are negative and there is no ascites.

IIC—Pelvic extension or implants like Stage IIA or IIB but with positive pelvic washings

Stage III—Cancer spread outside the pelvis to the abdominal area, including metastases to liver surface

IIIA—Tumor is grossly confined to the pelvis but with microscopic peritoneal metastases beyond pelvis to abdominal peritoneal surfaces or the omentum.

IIIB—Same as IIIA but with macro-scopic peritoneal or omental metastases beyond pelvis less than 2 cm in size.

IIIC—Same as IIIA but with peritoneal or omental metastases beyond pelvis, larger than 2 cm or lymph node metastases to inguinal, pelvic, or para-aortic areas.

Stage IV—Metastases or spread to the liver or outside the peritoneal cavity to areas such as the chest or brain.

Low malignant potential (LMP) ovarian cancer is a disease, in which precancerous cells (cells that may or that are likely to become cancerous) exist, but these tumors are characterized by the absence of ovarian stromal invasion, and yet they retain the ability to metastasize. LMP tumors are defined as cystadenomas with proliferative activity of the epithelial cells and nuclear abnormalities, but with no infiltrative destructive growth, and they seem to be a distinct class of tumors, usually do not advance to malign carcinomas.

The term “at risk of” a disease such as malignant ovarian tumor disease as used herein shall refer to a subject that potentially develops such a disease or already suffers from such a disease at various stages, including the early stage and advanced disease state, particularly associated with malignant tumors.

The risk determination to diagnose a malignant ovarian tumor is particularly important in a subject, where the malignancy has not yet been diagnosed. This risk determination therefore includes early stage diagnosis. Preferably those patients are tested for the biomarkers according to the invention, before an ovarian tumor is detected, or before malignancy has proven by biopsy.

Healthy patients are currently not tested for any tumor disease biomarkers in the absence of any detectable tumor. However, there are patients, who have the potential to develop malignant ovarian tumor disease because of a genetic predisposition. Antecedent diseases, such as cancer, or benign tumors or certain medical treatment would also increase the risk of developing solid tumors and associated disease conditions. Several risk factors for ovarian tumors that classify a high cancer risk have been identified so far, among them familial history of breast, uterus, colon or ovarian cancer, BRCA1 or BRCA2 gene mutations, hormonal therapies, never pregnant, probably obesity and use of fertility drugs.

The early detection of a malignant ovarian tumor disease is essential in the patient population that is already classified as high-risk patients. It is thus preferred to test a patient population according to the invention, which is already classified as risk patients.

In particular, the inventive method allows the early stage determination of the ovarian tumor disease or respective risk stages, e.g. to distinguish between low, medium and high risk patients.

In advanced cancer disease or minimal residual disease the risk of metastases can be high, which is usually associated with poor prognosis. Thus, the risk determination according to the invention particularly refers to the prognosis of a subject to develop cancer and/or the prognosis of a cancer patient, and in particular to the determination of the metastatic potential, such as tumors with low, medium or high metastatic potential.

In specific aspects of the invention, the method is a non-invasive method for the in vitro or ex vivo diagnosis of the malignant ovarian tumor disease; in particular the invention provides for the non-invasive, non-surgical method for detection, diagnosis, monitoring, or prediction of ovarian cancer or onset of ovarian cancer in a patient.

The invention preferably contemplates a gene expression profile comprising the multimarker panel according to the invention. This profile provides a highly sensitive and specific test with both high positive and negative predictive values permitting diagnosis and prediction of the patient's risk of developing cancer or the risk of developing metastatic disease.

Thus, the present invention specifically provides for the inventive gene signature derived from the expression in PBCs for the early diagnosis of ovarian cancer with high specificity and sensitivity. LMP tumors and cystadenomas are possibly diagnosed as benign tumors or not diagnosed as malignant tumors.

The preferred specificity of the method according to the invention is at least 99%, The sensitivity preferably is at least 70%, preferably at least 74%, specifically ranging between 85% and 90%, at a high specificity, such as the specificity of at least 99%. Therefore, a highly reliable test for diagnosing ovarian cancer, even in the early stages FIGO I and/or II is provided.

According to an example peripheral blood mononuclear cells were isolated from epithelial ovarian cancer (EOC) patients and healthy blood donors as controls and used a whole genome transcriptomics approach (Applied Biosystems Human Genome Survey microarrays V2.0) to find gene expression patterns to discriminate between ovarian cancer patients and healthy controls or patients with non-malignant diseases. Finally a predictive model was built from a large cohort of patients and controls using RT-qPCR derived expression values of selected gene panels, which were validated by means of the bootstrap 0.632+ cross-validation method.

In particular, upon comparing the expression of 32,000 genes in PBCs from 44 EOC patients and 19 controls by means of microarray analysis, three uncorrelated shrunken centroid models were selected, comprised of 7, 14, and 6 genes. These 27 genes were evaluated by RT-qPCR. A second selection using significance analysis of microarrays yielded the biomarkers according to the invention which were finally used in a validation step employing 343 samples (90 healthy controls, 6 cystadenoma, 8 low malignant potential (LMP) tumor, 19 FIGO I/II, and 220 FIGO III/1V EOC patients). The final model was built by LASSO logistic regression and cross-validated by bootstrap.632+.

It surprisingly turned out that an unexpected high specificity and selectivity could be obtained even in early stage cancer diagnosis. The bootstrap validated model showed an area under the ROC curve (AUC) of 0.967 and a classification error of 16.4%. This corresponds to a sensitivity of 88% at specificity of 99%. FIGO I/II patients were diagnosed with a sensitivity of 74% at specificity of 99% (AUC=0.905). PBCs from patients with cystadenomas and LMP tumors were not significant different from PBCs of healthy controls (AUC=0.658, P=0.058), whereas malignant tumors were distinguished from benign or LMP tumors with a sensitivity of 87% at specificity of 95% (AUC=0.939).

The analytical system to determine the expression of the multimarker gene panel according to the invention typically employs a detection system.

The term “detect” or “detecting” includes assaying, imaging or otherwise establishing the presence or absence of the target biomarker. The level of biomarkers or amount of biomarkers is herein understood to refer to the respective polypeptides or nucleotide sequence, including variants such as splice variants, subunits thereof, or reagent bound targets.

The target biomarker is preferably determined by testing for the respective polypeptides and/or polynucleotides indicative of marker expression. The expressed marker is detectable e.g. as polynucleotide, like mRNA, or expressed polypeptide or protein. The comparison with the reference value should be of the same sample type. Thus, the reagents preferably comprise ligands specifically binding to the biomarker polypeptide or gene or genetic marker, e.g. comprising a plurality of respective polypeptides, genes or polynucleotides. Ligands are herein understood as marker specific moieties.

Preferably the reagents are provided in suitable kits. Therefore, the invention also relates to kits for carrying out the methods of the invention.

The invention specifically contemplates the methods, compositions, and kits described herein using the inventive markers and further additional markers associated with ovarian or other epithelial cancer or used as normalization or control markers. The methods described herein may be modified by including reagents to detect the inventive markers and optionally additional markers, including polynucleotides for the markers.

Marker specific moieties are substances which can bind to or detect at least one of the markers for a detection method described above and are in particular marker nucleotide sequence detecting tools or marker protein specific antibodies, including antibody fragments, such as Fab, F(ab), F(ab)′, Fv, scFv, or single chain antibodies. The marker specific moieties can also be selected from marker nucleotide sequence specific oligonucleotides, which specifically bind to a portion of the marker sequences, e.g. mRNA or cDNA, or are complementary to such a portion in the sense or complementary anti-sense, like cDNA complementary strand, orientation.

According to a specific aspect there is provided a set of reagents to determine the biomarker panel according to the invention. The preferred set includes diagnostic tools, such as ligands to specifically bind the gene expression products or amplification products, e.g. through ligand affinity binding or hybridization of a nucleotide sequence. Thus, the preferred set employs primers to perform a PCR analysis to amplify each of the marker sequences of the inventive biomarkers, and means to detect or visualize the amplification products, including hybridizing probes.

Further preferred means include affinity ligands to bind the respective polypeptides, such as receptors, antibodies, antibody fragments, peptides or proteins.

The preferred ligands may be attached to solid surfaces to catch and separate the marker or PBC in the sample, optionally together with a label. Biological assays require methods for detection, and one of the most common methods for quantitation of results is to conjugate a detectable label to a protein or nucleic acid that has affinity for one of the components in the biological system being studied. Detectable labels may include molecules that are themselves detectable (e.g., fluorescent moieties, electrochemical labels, metal chelates, etc.) as well as molecules that may be indirectly detected by production of a detectable reaction product (e.g., enzymes such as horseradish peroxidase, alkaline phosphatase, etc.) or by a specific binding molecule which itself may be detectable (e.g., biotin, digoxigenin, maltose, oligohistidine, 2,4-dintrobenzene, phenylarsenate, ssDNA, dsDNA, etc.).

In particular aspects of the invention, the methods described herein utilize the multimarker panel placed on a microarray so that the expression status of each of the markers is assessed simultaneously. In an embodiment, the invention provides a microarray as a diagnostic tool comprising a defined set of ligands to the marker genes. The preferred microarray is provided on a single carrier that includes the means to react with the inventive biomarkers, which are preferably spatially separated. The reaction products may then be separately detected in a one-step procedure.

In preferred embodiments, the mRNA concentration of the markers is determined. To this extent, mRNA of the sample can be isolated, if necessary, after adequate sample preparation steps, e.g. PBC enrichment for white blood cells and/or lysis, and detected with marker specific probes, in particular on a microarray with or without amplification, e.g. employing primers for PCR-based detection methods, such as PCR extension labelling with probes specific for a portion of the marker mRNA.

According to a preferred embodiment, the invention provides a method for determining the risk of malignant ovarian tumor disease in a subject comprising

(a) contacting a PBC sample, optionally employing a PBC fraction obtained from said subject with one or more test oligonucleotides, each specifically hybridizing to a marker of the inventive panel, and optionally one or more internal control oligonucleotides, each specifically hybridizing with a control marker selected from the group consisting of RPL21, RPL9 and SH3BGRL3,

(b) detecting in the sample the level of reaction products of hybridization,

(c) optionally normalizing the level of reaction products of the test oligonucleotides by comparing with the reaction products of the internal control oligonucleotides,

(d) comparing the level of reaction products of the test oligonucleotides with a reference level or predetermined cut-off value, and therefrom diagnosing the malignant ovarian tumor disease in the subject.

Within certain preferred embodiments, the amount of mRNA is detected via polymerase chain reaction using, for example, oligonucleotide primers that hybridize to a marker gene, or complements of such polynucleotides. When using mRNA detection, the method may be carried out by combining isolated mRNA with reagents to convert to cDNA according to standard methods and analyzing the products to detect the marker presence in the sample. Within other embodiments, the genomic nucleic acid may be analyzed for the specific marker expression.

Therefore marker specific tools, such as primers for PCR amplification and probes to hybridize with the relevant nucleotide sequence can be used, which are commercially available, e.g. from Applied Biosystems (Foster City, Calif., USA).

In further embodiments the amount of a marker or any combination thereof is determined by the polypeptide or protein concentration of the marker(s), e.g. using marker specific ligands. The binding event can, e.g., be detected by competitive or non-competitive methods, including the use of labelled ligand or marker specific moieties or labelled competitive moieties, e.g. including a labelled marker standard, which compete with marker proteins for the binding event. If the marker specific ligand is capable of forming a complex with the marker, the complex formation indicates expression of the markers in the sample.

In particular, the invention relates to a method for diagnosing and monitoring malignant ovarian tumor disease in a patient by quantitating a marker in a PBC sample, optionally employing a PBC fraction, from the patient comprising

(a) reacting the sample with one or more binding agents, each specifically binding to a marker of the multimarker panel according to the invention, which binding agent is directly or indirectly labelled with a detectable substance, and

(b) detecting the detectable substance.

The preferred method employs an immunoassay. In general, immunoassays involve contacting a sample potentially containing a biomarker of interest with at least one immunoligand that specifically binds to the marker. Alternatively, competitive immunoassays employing binding moieties that compete with the binding of the marker to its ligand may be employed. A signal is then generated indicative of the presence or amount of complexes formed by the binding of polypeptides in the sample to the ligand. The signal is then related to the presence or amount of the marker in the sample. Immunoassays and respective tools for determining the markers of the inventive multimarker panels are well-known in the art or can be prepared using well-known techniques.

In particular, the ligands with specificity to bind the individual polypeptides or proteins of the inventive biomarkers, such as specific antibodies, can be prepared according to standard techniques or obtained from commercial sources, such as supplier of antibodies, e.g. Abcam plc (330 Cambridge Science Park, Cambridge, CB4 0FL, UK), Santa Cruz Biotechnology, Inc. (2145 Delaware Avenue, Santa Cruz, Calif. 95060, USA), LifeSpan Biosciences, Inc. (2401 Fourth Avenue, Suite 900, Seattle, Wash. 98121, USA), Novus Biologicals, LLC (8100 Southpark Way, A-8, Littleton, Colo. 80120, USA).

Preferably the differential marker expression is determined by comparing the expression to the control of healthy subjects or patients suffering from a benign tumor.

Reference values for the biomarker determination are preferably obtained from a control group of subjects with normal expression of said biomarker, or a biomarker expression, that is associated with the disease condition, such as disease stages, which represents the appropriate reference value. In a particular aspect, the control comprises material derived from a pool of samples from normal subjects. The normal level of a biomarker may be determined in samples of the same type obtained from control subjects.

The reference values are typically calculated from standard deviations of the mean average marker expression in healthy subjects. If more than one marker is detected, the comparison is made to each single reference value for each marker in the reference itself. The malignant ovarian tumor disease may be diagnosed if the amount of the biomarker or the combination of markers exceeds the cut-off value determined according the preferred specificity of 99% or a sensitifity of 99%. The risk score is preferably calculated according to a logistic regression model, such as provided in Table 3 below. As a reference the value of subjects not suffering from such tumor, preferably being subjects from a control group or healthy subjects, is used. If at least two biomarkers of the panel according to the invention differ from the reference value, the risk is considered to be increased as well.

The marker level can also be compared to a threshold, e.g. a cut-off concentration and the risk of the ovarian tumor development is determined from such comparison; wherein the biomarker concentration above (upregulated) or below (downregulated) the reference value is predictive of tumor development in the patient.

Thus, the preferred method according to the invention comprises the step of comparing the biomarker level with a predetermined standard or cut-off value, which is preferably at least 25% different from the standard, more preferred at least 40% or 50% different, but can also be at least 100% different.

The higher the fold difference to the reference value, the higher is the patient's risk of developing or the chance of having such ovarian tumor and cancer. The typical gene signature of the multimarker panel according to the invention indicates, for example, special treatment of the patient, using appropriate medication or further diagnostic techniques, such as imaging and surgical interventions. The method of the invention can thus be used to evaluate a patient before, during, and after medical treatment.

Types of cancer treatment that are used as adjuvant therapy include chemotherapy, hormone therapy, radiation therapy, immunotherapy or targeted therapy. Following first line chemotherapy, for instance, the cancer patient can be determined for the metastatic potential to decide about a second line adjuvant treatment.

The preferred method of determining the gene signature of a PBC sample of a patient employing the multimarker panel according to the invention may also be particularly useful to monitor the marker level during such treatment.

The present invention is further illustrated by the following examples without being limited thereto.

### EXAMPLES

### Example 1

### Materials and Methods

Patients and Controls

In total, blood from 239 epithelial ovarian cancer (EOC) patients and 104 controls (90 healthy blood donors and 14 patients with benign tumors (adenoma) or low malignant potential (LMP) tumors (borderline) were enrolled in this retrospective study (Table 2). All blood samples from FIGO II, III, and IV epithelial ovarian cancer patients were collected in the course of the EU-project OVCAD within two days prior to surgery (Charité, Berlin Medical University, Germany n=86, University Clinic Hamburg-Eppendorf, Germany n=43, Medical University of Innsbruck, Austria n=11, Katholieke Universiteit Leuven, Belgium n=52, Medical University of Vienna, Austria n=35), all other blood samples were from patients or from blood donors from the Medical University of Vienna. Informed consent for the scientific use of biological material was obtained from all patients and blood donors in accordance with the requirements of the ethics committee of the institutions involved.

TABLE 2
Characteristics of EOC patients, patients with benign or low
malignant potential tumors, and healthy blood donors as controls
(Healthy vs. EOC I, cohort 1 and Healthy vs. EOC II, cohort 2).

| | | | | Age ± SD | Range |
|---|---|---|---|---|---|
| Cohort 1 | Typ | Number | FIGO | [years] | [years] |
| Controls | Healthy | 90 | n.a. | 46.7 ± 16.8 | 19-83 |
| Benign disease | Adenoma | 6 | n.a. | 57.3 ± 8.5 | 45-66 |
| Low malignant | Borderline | 8 | n.a. | 60.0 ± 18.6 | 32-92 |
| potential | tumor | | | | |
| (LMP) | | | | | |
| Malignant | Ovarian | 19 | FIGO I/II | 55.5 ± 16.7 | 15-85 |
| disease | cancer | | | | |
| Malignant | Ovarian | 220 | FIGO | 58.6 ± 11.8 | 18-83 |
| disease | cancer | | III/IV | | |
| Cohort 2 | | | | | |
| Controls | Healthy | 30 | n.a. | | |
| Benign disease | Adenoma | 35 | n.a. | | |
| Malignant | Ovarian | 14 | FIGO I/II | | |
| disease | Cancer | | | | |
| (overlapping | Ovarian | 210 | FIGO | | |
| with cohort 1) | Cancer | | III/IV | | |

PBC Isolation and Total RNA Preparation

A peripheral blood fraction enriched in PBC, specifically the PBC fraction enriched in lymphocytes and granulocytes, and depleted from epithelial cells were isolated from EDTA-blood according to Brandt and Griwatz (Clin Chem 11996; 42: 1881-2). A buoyant density centrifugation gradient was employed and the granulocyte and lymphocyte fraction was further used for determining the markers of the multimarker panel. Total RNA was isolated using the RNeasy Mini kit (QIAGEN, Venlo, Netherlands) and quality-checked with the Agilent's 2100 Bioanalyzer (Agilent Technologies, Santa Clara, Calif., USA). The RNA-quantity was measured spectrophotometrically.

Microarray Analysis and Preselection

Whole genome gene expression analysis was performed on single channel Applied Biosystems Human Genome Survey microarrays V2.0 (Applied Biosystems, Foster City, Calif., USA) containing 32,878 probes representing 29,098 genes. Two μg total RNA from 44 ovarian cancer patients and 19 age-matched controls (mean 60.8±13.7 years and 61.7±12.9 years, respectively) were labeled with the NanoAmp RT-IVT Labeling Kit and hybridized to the microarrays for 16 hours at 55° C. After washing and visualization of bound digoxigenin-labeled cRNAs with the Chemiluminescence Detection Kit according to the manufacturer's instructions (Applied Biosystems), images were read with the 1700 Chemiluminescent Microarray Analyzer (Applied Biosystems). Raw expression data, signal-to-noise ratios and quality-flags delivered from the Applied Biosystems Expression System software were further processed using Bioconductor's ABarray package (Gentleman R C et al., Genome Biol. 2004; 5(10):R80. Bioconductor: open software development for computational biology and bioinformatics). In brief, raw expression values were log₂ transformed, and measurements with quality indicator flag values greater than 5000 were set missing. For inter-array comparability, data were quantile-normalized and missing values imputed with 10-nearest neighbors imputation. As there are extensive batch-effects with this type of microarrays and all microarrays with controls were from one batch which was different from the batches of the microarrays of the ovarian cancer patients, an extensive pre-filtering of probes was performed. Firstly, 13,520 probeIDs which exhibited a signal-to-noise ratio less than 2 in at least 50% of the two pooled groups (patients with malignant disease and non-malignant controls) were excluded (19,358 probeIDs were remaining). Secondly, 10,125 probeIDs assumed to be potentially affected by batch-effects were excluded, resulting in remaining 9,233 probeIDs. The existence of these batch-effects was concluded from a microarray experiment, which comprised more than 200 microarrays and included all batches used in the present study. In a last filtering step 205 probeIDs with fold-changes >3 between both groups were selected. Three further genes were eliminated due to non-available TaqMan® Assay-on-Demand probes and primer sets (Applied Biosystems). From the remaining 202 probeIDs three consecutive predictive models were built using the uncorrelated shrunken centroids approach (USC) (Yeung K Y, Bumgarner R E. Genome Biol 2003; 4: R83), implemented in the MultiExperiment Viewer (MeV) (Saeed Al et al. Methods Enzymol 2006; 411: 134-93). This method selects uncorrelated genes which best discriminate the two groups in internal cross-validation. Since the method picks only one gene from a group of several highly correlated genes, and this selection may be arbitrarily affected by small-sample variation, we repeated the method twice each time excluding the genes found in the previous step. This iterative approach leads to a richer set of candidate genes for further analyses.

Evaluation of Microarray Results

The microarray gene expression measurements of the selected genes were validated by real time PCR. cDNA was synthesized from 1 μg total RNA using the M-MLV reverse transcriptase (Promega, Madison, Wis., USA) and a random nonamer primer. For normalization three stably expressed genes were selected from all 63 microarrays and all genes with signal-to-noise ratios greater than 3 in all samples (8,318 probeIDs): RPL21 (Ribosomal protein L21, Assay-on-Demand TaqMan® probe: Hs03003806_g1), RPL9 (Ribosomal protein L9, Hs01552541_g1), and SH3BGRL3 (SH3 domain-binding glutamic acid-rich-like protein 3, Hs00606773_g1), with coefficients of variation (CV) of 0.014, 0.012, and 0.014, respectively. The geometric mean of the RT-qPCR values of these three normalizers was calculated for each sample and this normalizing sample-specific constant was subtracted from each measurement of sample to obtain normalized (delta-CT) values. Delta-CT values were transformed to log₂-expression values by multiplying the values with −1.

Statistical Analysis, Model Building, and Bootstrap Validation

The significance of the differences in mean age between the five clinically defined groups (Table 2) was calculated by the ANOVA test, followed by a Tukey's post hoc test. The significance of the up- or down-regulation of the expression of the 13 genes between healthy controls and patients with malignant disease was calculated by the t test followed by correction for multiple testing by the Holm-Bonferroni method.

The log₂ expression values were compared between healthy and malignant tumor samples by the significance analysis of microarrays (SAM) procedure, employing the t test statistic and using R's samr package (Tusher V G et al. Proc Natl Acad Sci USA 2001; 98: 5116-21). Genes with q-values less than 0.15 were selected for model building and validation. In the last step, the genes selected by the SAM procedure were re-assessed by RT-qPCR in all 239 malignant, 90 healthy, and 14 low-malignant potential or benign samples.

Gene expression values were normalized as described above, and a L1 regularized logistic regression model, also known as LASSO, was estimated to obtain a model discriminating between the healthy and diseased groups (Park M Y, Hastie T. Journal of the Royal Statistical Society Series B-Statistical Methodology 2007; 69: 659-677). The misclassification error rate and the cross-validated receiver operating characteristic curve were estimated using the bootstrap 0.632+ cross-validation procedure (Efron B, Tibshirani R. Journal of the American Statistical Association 1997; 92: 548-560). The glmpath R package was used for computing the LASSO model.

Results

Starting from the 202 genes preselected as described above, three consecutive uncorrelated shrunken centroid (USC) models were built, comprised of 7, 14, and 6 genes, respectively. Expressions of these 27 genes were validated in 63 samples using RT-qPCR with corresponding Assay-on-Demand TaqMan® probes (Applied Biosystems, Foster City, Calif., USA) (Table 3) and a set of three stably expressed genes as normalizers, selected also from the microarray data. Seven of these 27 failed the validation step, because these genes showed no expressions in the 63 samples, indicating microarray artifacts or problems with the Assay-on-Demand TaqMan® probes (Table 3). A further selection step by Significance Analysis of Microarrays (SAM) selected 13 of the remaining 20 genes with q-values 0.15 (Table 3).

TABLE 3
Gene list of the 27 genes from the three USC-models, corresponding
Assay-on-Demand TaqMan ® probes, SAM-results from the
second selection step, and coefficients of the final LASSO logistic regression model.

| Genes | | | | SAM | |
|---|---|---|---|---|---|
| | Gene | Evaluation | | q-value | LASSO model |
| ProbeID | symbol | TaqMan ® probe | RT-qPCR | (≦0.15) | Coefficient |
| Model 1 | | | | | |
| 119290 | CFP | Hs00175252_m1 | yes | 0.13 | 1.241 |
| 182018 | NOXA1 | Hs01017917_m1 | yes | 0.09 | −0.888 |
| 184360 | RETNLB | Hs00395669_m1 | no | | |
| 212552 | ZNF546 | Hs00418908_m1 | no | | |
| 228089 | NEAT1 | Hs01008264_s1 | yes | 0.01 | 2.075 |
| 713562 | N/A (BC037918) | Hs00860048_g1 | yes | 0.01 | 0.035 |
| 10546171 | N/A | Hs01036865_m1 | no | | |
| Model 2 | | | | | |
| 105700 | AMZ1 | Hs00401010_m1 | no | | |
| 105743 | DIS3 | Hs00209014_m1 | yes | 0.10 | 1.177 |
| 109227 | ZNF419 | Hs00226724_m1 | yes | 0.08 | 0.145 |
| 110071 | CCR2 | Hs00356601_m1 | yes | 0.01 | 0.376 |
| 110496 | DYSF | Hs00243339_m1 | yes | 0.49 | not used |
| 118384 | HGS | Hs00610371_m1 | yes | 0.39 | not used |
| 136788 | ALX4 | Hs00222494_m1 | no | | |
| 142487 | B4GALT1 | Hs00155245_m1 | yes | 0.11 | −0.642 |
| 160314 | DBNL | Hs00429482_m1 | yes | 0.50 | not used |
| 161219 | MPP1 | Hs00609971_m1 | yes | 0.41 | not used |
| 161567 | PAPOLG | Hs00224661_m1 | yes | 0.01 | −0.454 |
| 162222 | PRIC285 | Hs00375688_m1 | yes | 0.09 | −1.794 |
| 223870 | CCL3L1 | Hs00824185_s1 | yes | 0.32 | not used |
| 224628 | ANKHD1 | Hs00226589_m1 | yes | 0.24 | not used |
| Model 3 | | | no | | |
| 115368 | AP2A1 | Hs00367123_m1 | yes | 0.15 | −0.199 |
| 157342 | C1orf63 | Hs00220428_m1 | yes | <0.01 | −0.230 |
| 177183 | RMI1 | Hs00227878_m1 | yes | 0.37 | not used |
| 204670 | GRM1 | Hs00168250_m1 | no | | |
| 205406 | OSM | Hs00171165_m1 | yes | 0.01 | −1.105 |
| 220229 | ASGR1 | Hs00155881_m1 | no | | |
| | | | | | Intercept: 6.320 |

Normalized RT-qPCR expression values of these 13 genes were determined from all 343 PBC samples. Three genes were significantly up-regulated in PBCs of 239 EOC patients compared to 90 healthy blood donors, NEAT1 and the transcript BC037918 (both non-coding RNAs) and CCR2 (Table 1). Three genes were not significantly different expressed in both groups (B4GALT1, DIS3 and ZNF419). All other genes were significantly down-regulated in the EOC patients compared to the controls (Table 1). In Table 4 the corresponding area under the receiver operating characteristic (ROC)-curve (AUC) values for all 13 genes using 90 healthy blood donors as controls and the 239 epithelial ovarian cancer patients are shown. The expression of five genes was associated with higher probability of EOC, two of them non-significantly (again DIS3 and ZNF419), and eight genes were negatively correlated with the probability of EOC. The latter were therefore inverted for the ROC curves and AUC estimations (cf. Table 4). Using LASSO logistic regression a predictive model was built to discriminate between healthy blood donors as controls and the 239 EOC patients. The model selected all 13 genes including the genes which were not significant in the univariate analyses (Table 3). CFP was the only gene whose predictive value changed from its negative direction in the univariate analysis to a positive contribution in the LASSO multivariate model. Table 4 refers to the cross-validated ROC curve of the LASSO model with an AUC of 0.967 (95% CI 0.956-0.987). This corresponds to a sensitivity of 88% (95% CI 83%-92%) at a specificity fixed at 99%. The bootstrap 0.632+ estimate of classification error was 16.4%.

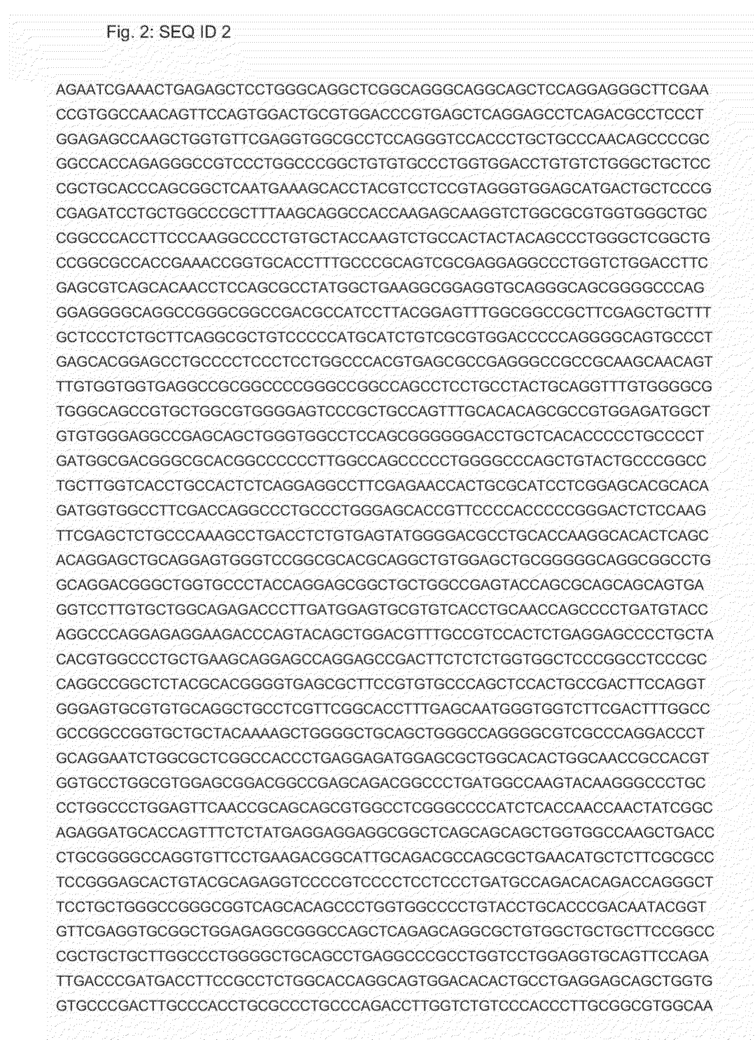

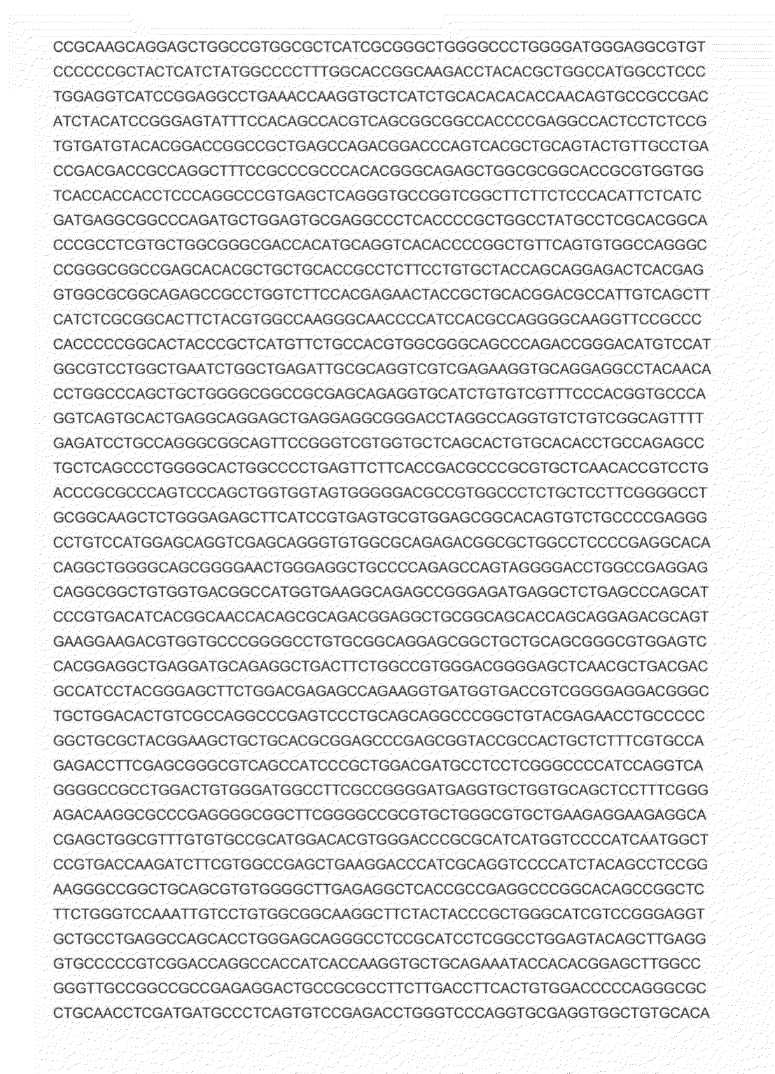

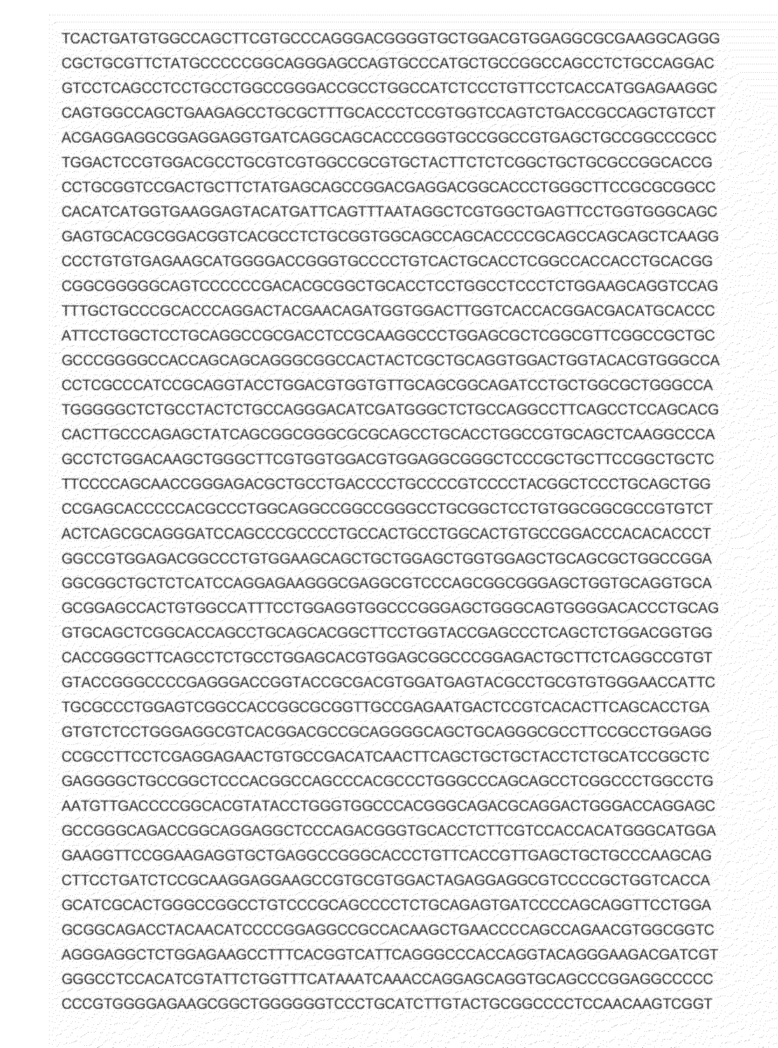

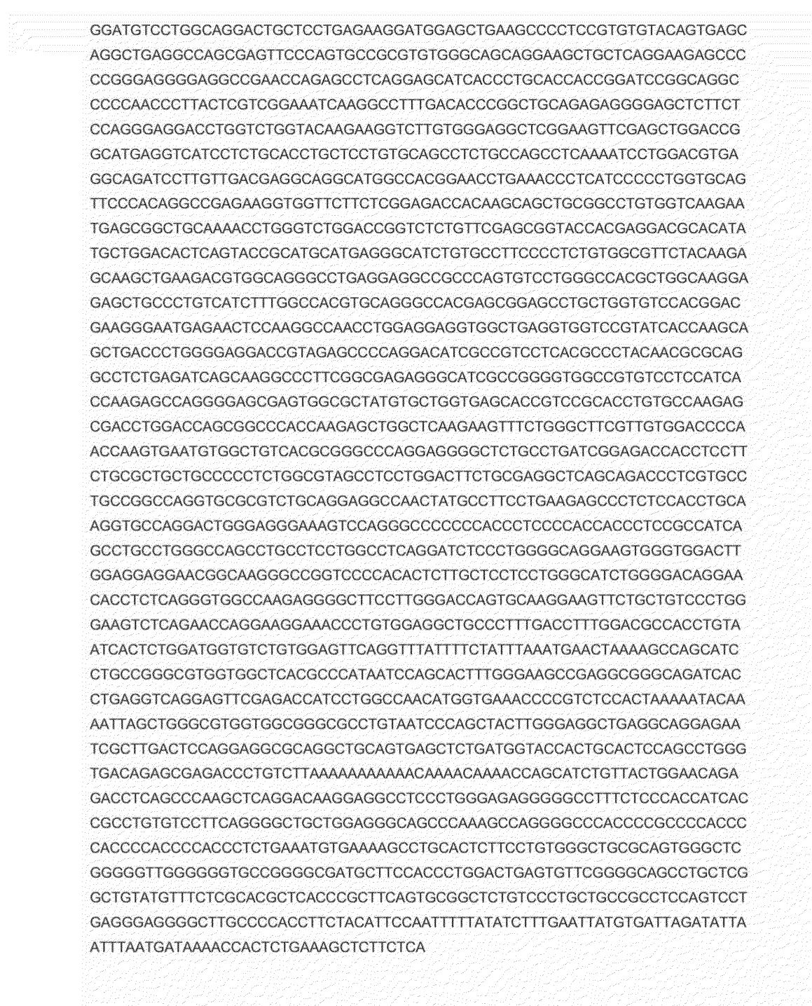

The same model discriminated FIGO I/II patients from controls with a sensitivity of 74% at a specificity fixed at 99% (AUC=0.905, 95% CI 0.781-1.000, Table 4). However, our model could not well discriminate between PBCs from healthy controls and patients with benign or low malignant potential tumors (AUC=0.658, p=0.058). Nevertheless, malignant tumors were distinguished from benign or LMP tumors with a sensitivity of 87% at a specificity fixed at 95% (AUC=0.939, 95% CI 0.902-0.976) (Table 4) and even FIGO I/II EOC tumors were different from benign or LMP tumors with an AUC of 0.853 (95% CI 0.719-0.987) (FIG. 2F, Table 3).

Since the healthy donors were significantly younger than the EOC patients (Table 2), we investigated whether the LASSO estimated risk score (i.e., the sum of each subject's gene expressions weighted by the LASSO model coefficients) was correlated to age of the donors or the patients. This was not the case, as confirmed by irrelevant correlation coefficients of the risk score with age of 0.083 (p=0.449) in healthy donors and 0.104 (p=0.111) in EOC patients.

The risk score was calculated as follows:

Risk-score=(1.241*CFP)+(−0.888*NOXA1)+(2.075*NEAT1)+(0.035*BC037918)+(1.177*DIS3)+(0.145*ZNF419)+(0.376*CCR2)+(−0.642*B4GALT1)+(−0.454*PAPOLG)+(−1.794*PRIC285)+(−0.199*AP2A1)+(−0.23*C1orf63)+(−1.105*OSM)+6.32

The LASSO model was used as described in Mee Young Park and Trevor Hastie (2007) L1 regularization path algorithm for generalized linear models. (J. R. Statist. Soc. B, 69, 659-677).

TABLE 4
Area under the Receiver Operating Characteristic curves (AUC) of the
13 single genes and the final LASSO model (Healthy vs EOC I).

| | | | Asymptotic 95% | |
|---|---|---|---|---|
| | | Asymptotic | Confidence Interval | |
| ProbeID | | Sig. | Lower | Upper |
| (Healthy vs. EOC I) | AUC | [p-value] | Bound | Bound |
| 105743 | 0.525 | 0.484 | 0.460 | 0.590 |
| 109227 | 0.541 | 0.249 | 0.475 | 0.608 |
| 110071 | 0.618 | 0.001 | 0.556 | 0.680 |
| 228089 | 0.822 | <0.001 | 0.778 | 0.866 |
| 713562 | 0.721 | <0.001 | 0.665 | 0.778 |
| inv115368 | 0.684 | <0.001 | 0.625 | 0.744 |
| inv119290 | 0.610 | 0.002 | 0.546 | 0.674 |
| inv142487 | 0.589 | 0.013 | 0.525 | 0.653 |
| inv157342 | 0.638 | <0.001 | 0.568 | 0.707 |
| inv161567 | 0.639 | <0.001 | 0.576 | 0.702 |
| inv162222 | 0.804 | <0.001 | 0.758 | 0.851 |
| inv182018 | 0.600 | 0.005 | 0.537 | 0.664 |
| inv205406 | 0.731 | <0.001 | 0.675 | 0.786 |
| LASSO model | | | | |
| Healthy vs. EOC | 0.971 | <0.001 | 0.956 | 0.987 |
| Healthy vs. FIGO I/II | 0.905 | <0.001 | 0.781 | 1.000 |
| Benign/LMP vs. EOC | 0.939 | <0.001 | 0.902 | 0.976 |
| Benign/LMP vs. FIGO I/II | 0.853 | 0.001 | 0.719 | 0.987 |

### Example 2

Using a different cohort of healthy and benign controls and an overlapping cohort of EOC patients (Table 2, cohort 2) the abundance of six proteins (MIF, prolactin, CA125, leptin, IGF2, and osteopondin) in plasma was determined using a test kit from Millipore (Milliplex Map kit, Cancer Biomarker Panel, Millipore Corp., MA, USA). The log-2 values of these six proteins were used in combination with the expressions of the 13 genes to build L1 (LASSO) and L2 (ridge) diagnostic models (using R package penalized 0.9-35) and the classification errors were estimated by bootstrap 0.632+ validation. In Table 5 area under the receiver operating characteristic curves for two combined models, two models only using 7 or 13 genes, respectively, and two models using only four or six proteins, respectively, are shown. In Table 6 the characteristics of the two combined models are shown in detail. The L1 model comprised of five gene expressions (228089: NEAT1; 713562: BC037918; 157342: C1orf63; 162222: PRIC285; 205406: OSM) and 5 serum protein values (0.63*228089+0.22*713562+0.35*157342−1.58*162222−0.54*205406−0.054*Ig2_MIF+0.471 g2_Prolactin+0.33*Ig2_CA125−0.551 g2_Leptin−0.47*Ig2_IGF2+4.91) showed a sensitivity of about 98% at a specificity fixed at 99.6%. The bootstrap 0.632+ estimated classification error was 3.1%. A model comprised of all 13 genes and 6 serum proteins (0.26*105743−0.01*109227−0.16*110071+0.70*228089+0.29*713562−0.36*115368+0.34*119290+0.12*142487+0.55*157342−0.27*161567−1.30*162222−0.09*182018−0.870370508*205406−0.24*Ig2_MIF+0.531 g2_Prolactin+0.39*Ig2_CA125−0.591 g2_Leptin−0.021 g2_Osteopondin−0.51*Ig2_IGF2+7.31) showed a sensitivity of about 96% at a specificity fixed at 99.6%. The bootstrap 0.632+ estimated classification error was 2.8%. Further models are shown in Table 7 with AUC values shown in Table 5.

TABLE 5
Area under the Receiver Operating Characteristic curves (AUC) of
further L1 and L2 models (Healthy vs EOC II).

| | | | Asymptotic 95% | |
|---|---|---|---|---|
| | | Asymptotic | Confidence Interval | |
| ProbeID | | Sig. | Lower | Upper |
| (Healthy vs. EOC II) | AUC | [p-value] | Bound | Bound |
| L1 5 expressions 5 | .998 | <.0001 | .994 | 1.000 |
| proteins | | | | |
| L2 13 expressions 6 | .998 | <.0001 | .995 | 1.000 |
| proteins | | | | |
| L1 7 expressions | .984 | <.0001 | .972 | .996 |
| L2 13 expressions | .987 | <.0001 | .976 | .997 |
| L1 4 proteins | .973 | <.0001 | .956 | .990 |
| L2 6 proteins | .973 | <.0001 | .956 | .989 |

TABLE 6
Characteristics of the combined expression and protein models.

| Model | L1 lasso penalty | L2 ridge penalty |
|---|---|---|
| Blood expression values | 5 genes | 13 genes |
| Plasma protein values | 5 proteins | 6 proteins |
| AUC (Area under the ROC curve) | 0.998 | 0.998 |
| AUC (Early Stages, FIGO I/II) | 0.976 | 0.979 |
| Specificity (set) | 99.6% | 99.6% |
| Sensitivity | 97.8% | 95.6% |
| Classification error | 3.1% | 2.8% |
| (bootstrap .632+) | | |

TABLE 7
L1 and L2 prognostic models built with either only gene
expression values or serum protein values

| | Coefficients | | | |
|---|---|---|---|---|
| | L1-7 | L2-13 | L1-4 | L2-6 |
| Gene/protein | expressions | expressions | proteins | proteins |
| 105743 | 0.02 | 0.63 | | |
| 109227 | | 0.05 | | |
| 110071 | 0.17 | 0.26 | | |
| 228089 | 0.91 | 0.99 | | |
| 713562 | 0.34 | 0.34 | | |
| 115368 | | −0.18 | | |
| 119290 | | 0.28 | | |
| 142487 | | 0.15 | | |
| 157342 | 0.36 | 0.64 | | |
| 161567 | | −0.56 | | |
| 162222 | −1.34 | −1.34 | | |
| 182018 | | −0.38 | | |
| 205406 | −0.83 | −1.05 | | |
| Ig2_MIF | | | | 0.09 |
| Ig2_Prolactin | | | 0.67 | 0.62 |
| Ig2_CA125 | | | 0.71 | 0.67 |
| Ig2_Leptin | | | −0.32 | −0.35 |
| Ig2_Osteopondin | | | | 0.05 |
| Ig2_IGF2 | | | −0.31 | −0.35 |
| Intercept | 3.93 | 4.90 | −2.71 | −2.77 |

Discussion

Peripheral blood cells, such as the white blood cell fraction seem to be an informative blood compartment for early diagnosis of epithelial ovarian cancer. The diagnostic power is similar to marker panels derived from serum proteins. For the first time it has proven that the expression pattern of five genes, optionally together with further genes such as the selected genes in PBCs can discriminate healthy controls from epithelial ovarian cancer patients, e.g. with a validated classification error of 16.4%. We reached a sensitivity of at least 95% at a specificity fixed at at least 99%, which could be high enough for use as a predictive test in high-risk individuals or as second test in combination with a CA-125 based screening approach.

Furthermore, our model can distinguish benign or low malignant potential tumors from malignant tumors with a rather high sensitivity and specificity (at least 87% and 95%, respectively), probably useful for differential diagnosis, which has a tremendous impact on treatment needs and prognosis.

Only Zhang at al. (reference above) has tested their multi-marker serum panel for the discriminatory potential between benign or low malignant potential tumors and malignant tumors, with specificities and sensitivities in the range of 33%-45% and 33%-50%, respectively. As serum proteins used for serum based tests are thought to be derived from the tumors itself, it was surprising that differences in PBC expressions, representing the status of the immune system, are driven by the malignant processes more than solely by tumor masses. Discrimination between benign and malign tumors turned out to be easier with PBC expression patterns than with serum protein patterns.

The functional specifications of the five genes, and in particular further genes selected from the 13 genes panel are widespread among the pool of functional clusters and pathways, which is not a big surprise given the extensive pre-filtering of our transcriptomics data (Table 1). Nevertheless, four genes are involved directly in inflammatory response and the immune system (B4GALT1, CCR2, CFP, and OSM), and two of them in the JAK/STAT pathway (CCR2 and OSM), known to be a common signaling pathway used by many cytokines. Two genes seem to be non-coding RNAs (NEAT1 and transcript BC037918), presumably involved in regulation of transcription. The other protein functions are completely incoherent: one is a zinc-finger protein (ZNF419) of unknown function, one a poly(A) polymerase (PAPOLG), one a co-activator for several nuclear receptors like PPARA, PPARG, TR-beta-1, ER-alpha, and RXR-alpha (PRIC285), one an activator of catabolic NADPH oxidases (NOXA1), one is an RNase enzyme and can be part of the exosome complex (DIS3), and one is involved in the assembly of clathrin coated vesicles (AP2A1). From one transcript, C1orf63, no homologue protein is known.

The PBC-expression based test according to the invention can be further improved by serum protein based tests, such as the commercial available OVA1™ test offered by Quest Diagnostics in the USA or the protein panel described by Visintin et al. (Visintin I et al. Clin Cancer Res 2008; 14: 1065-72), which could allow real detection of ovarian cancer with a near 100% sensitivity and specificity.

1. A method of diagnosing a malignant ovarian tumor disease in a subject, which comprises:
providing a sample of peripheral blood cells (PBC) of the subject,
measuring the expression of a multimarker gene panel comprising at least NEAT1, BC037918, C1orf63, PRIC285, and OSM, and
comparing the expression of the multimarker gene panel to a reference value, the differential expression being indicative of a malignant ovarian tumor.

2. The method of claim 1, wherein the sample is obtained from a blood fraction enriched in white blood cells, including granulocytes and optionally lymphocytes.

3. The method of claim 1, wherein the sample is obtained from a blood fraction from which epithelial cells have been depleted.

4. The method of claim 1, wherein the expression of at least one further gene selected from the group consisting of B4GALT1, CCR2, CFP, ZNF419, PAPOLG, NOXA1, DIS3 and AP2A1 is determined.

5. The method of claim 1, wherein the expression of at least NEAT1, BC037918, C1orf63, PRIC285, OSM, DIS3 and CCR2 is determined.

6. The method of claim 1, wherein the expression of at least NEAT1, BC037918, C1orf63, PRIC285, OSM, B4GALT1, CCR2, CFP, ZNF419, PAPOLG, NOXA1, DIS3 and AP2A1 and is determined.

7. The method of claim 1, wherein the expression of at least one further gene selected from the group consisting of RPL21, RPL9 and SH3BGRL3 is determined as an internal control.

8. The method of claim 1, wherein nucleic acid expression of the multimarker gene panel is determined, preferably the expression of mRNA.

9. The method of claim 1, further comprising the step of performing a further screening method for serum proteins or proteins from other bodily fluids which are indicative of ovarian cancer.

10. The method of claim 9, wherein the nucleic acid or protein expression of the serum proteins or proteins from other bodily fluids is determined.

11. The method of claim 1, further comprising the step of performing another diagnostic method that determines clinical parameters which are indicative of ovarian cancer, such as transvaginal ultrasound.

12. The method of claim 9, wherein said serum proteins are selected from the group consisting of CA-125, HE4, CEA, VCAM-1, MIF, Leptin, Prolactin, OPN, IGF-II, apolipoprotein A1, transthyretin or truncated transthyretin, ITIH4, hepcidin, β2-microglobulin, transferrin, CTAP3 and inter-alpha-trypsin inhibitor heavy chain H4 or a cleavage fragment thereof.

13. The method of claim 1, wherein the expression of at least NEAT1, BC037918, C1orf63, PRIC285, OSM, MIF, Prolactin, CA125, Leptin and IGF-II is determined.

14. The method of claim 1, wherein the expression of at least NEAT1, BC037918, C1orf63, PRIC285, OSM, B4GALT1, CCR2, CFP, ZNF419, PAPOLG, NOXA1, DIS3, AP2A1, and further at least MIF, Prolactin, CA125, Leptin, IGF-II and OPN is determined.

15. (canceled)

16. The method of claim 1, wherein the malignant ovarian tumor is predicted at an early stage, in particular the FIGO I/II stage, with a specificity of at least 99%.

17. The method of claim 1, wherein said malignant ovarian tumor is differentiated from a benign or low malignant potential tumor.

18. A set of reagents to determine the expression of a multimarker panel in a PBC fraction at least consisting of NEAT1, BC037918, C1orf63, PRIC285, OSM, and optionally at least one of B4GALT1, CCR2, CFP, ZNF419, PAPOLG, NOXA1, DIS3 and/or AP2A1, comprising reagents to determine the individual markers of said panel.

19. The set of claim 17, wherein the multimarker panel comprises less than 100 individual markers, preferably less than 50 individual markers.

20. The set of claim 18, further comprising means to prepare a PBC fraction.

21. The set of claim 18, wherein the reagents determine mRNA of the individual markers.

22. The set of claim 18, further comprising reagents for determining the expression of at least one of the RPL21, RPL9, and/or SH3BGRL3 genes as an internal control.

23. The set of claim 18, further comprising primers to perform the RT-qPCR analysis of the individual markers.

24. The set of claim 18, further comprising probes to specifically hybridise with gene transcription products of the individual markers.

25-26. (canceled)

27. A method of diagnosing malignant ovarian tumor disease in a subject, comprising the steps of:
determining the expression level of a marker for malignant ovarian tumor disease;
determining the expression level of at least one of the RPL21, RPL9 and/or SH3BGRL3 genes in a PBC fraction of the subject; and
comparing the expression level of the marker to the expression level of at least one of the RPL21, RPL9 and/or SH3BGRL3 genes as an internal control to quantify the expression of the marker.
